(19) United States
(12) Patent Application Publication
(10) Pub. No.: US 2017/0002041 A1
(43) Pub. Date: Jan. 5, 2017

(54) NOVEL ULTRASHORT HYDROPHOBIC PEPTIDES THAT SELF-ASSEMBLE INTO NANOFIBROUS HYDROGELS AND THEIR USES

(71) Applicant: AGENCY FOR SCIENCE, TECHNOLOGY AND RESEARCH, Connexis (SG)

(72) Inventors: Charlotte Hauser, Singapore (SG); Yihua Loo, Singapore (SG)

(73) Assignee: Agency for Science, Technology and Research, Connexis (SG)

(21) Appl. No.: 15/039,922

(22) PCT Filed: Nov. 28, 2014
(86) PCT No.: PCT/SG2014/000568

§ 371 Date: May 27, 2016

(30) Foreign Application Priority Data
Nov. 30, 2013 (SG) 201308890-1

(51) Int. Cl. C07K 7/06; C12N 5/00; C07K 5/062; A61L 27/52; C07K 5/103; C07K 5/083

(52) U.S. Cl. CPC C07K 7/06; C07K 5/101; C07K 5/0808; C07K 5/06052; C07K 5/06034; A61L 27/52; C12N 5/0062; C12N 5/0068; A61L 2400/06; A61L 2430/34; A61K 38/00

(57) ABSTRACT

The present invention relates to hydrophobic peptides and/or peptidomimetics capable of forming a (nanofibrous) hydrogel and hydrogels comprising said hydrophobic peptides and/or peptidomimetics and to various uses, such as in regenerative medicine, injectable therapies, delivery of bioactive moieties, wound healing, 2D and 3D synthetic cell culture substrate, biosensor development, biofunctionalized surfaces, and biofabrication.

### FIELD OF THE INVENTION

The present invention relates to hydrophobic peptides and/or peptidomimetics capable of forming a (nanofibrous) hydrogel and hydrogels comprising said hydrophobic peptides and/or peptidomimetics and to various uses, such as in regenerative medicine, injectable therapies, delivery of bioactive moieties, wound healing, 2D and 3D synthetic cell culture substrate, biosensor development, biofunctionalized surfaces, and biofabrication.

### BACKGROUND OF THE INVENTION

Self-assembly is an elegant and expedient “bottom-up” approach towards designing ordered, three-dimensional and biocompatible nanobiomaterials. Reproducible macromolecular nanostructures can be obtained due to the highly specific interactions between the building blocks. These intermolecular associations organize the supramolecular architecture and are mainly non-covalent electrostatic interactions, hydrogen bonds, van der Waals forces, etc. Supramolecular chemistry or biology gathers a vast body of two or three dimensional complex structures and entities formed by association of chemical or biological species. These associations are governed by the principles of molecular complementarity or molecular recognition and self-assembly. The knowledge of the rules of intermolecular association can be used to design polymolecular assemblies in form of membranes, films, layers, micelles, tubules, gels for a variety of biomedical or technological applications (J.-M. Lehn, Science, 295, 2400-2403, 2002).

Peptides are versatile building blocks for fabricating supramolecular architectures. Their ability to adopt specific secondary structures, as prescribed by amino acid sequence, provides a unique platform for the design of self-assembling biomaterials with hierarchical three-dimensional (3D) macromolecular architectures, nanoscale features and tuneable physical properties (S. Zhang, Nature Biotechnology, 21, 1171-1178, 2003). Peptides are for instance able to assemble into nanotubes (U.S. Pat. No. 7,179,784) or into supramolecular hydrogels consisting of three dimensional scaffolds with a large amount of around 98-99% immobilized water or aqueous solution. The peptide-based biomaterials are powerful tools for potential applications in biotechnology, medicine and even technical applications. Depending on the individual properties these peptide-based hydrogels are thought to serve in the development of new materials for tissue engineering, regenerative medicine, as drug and vaccine delivery vehicles or as peptide chips for pharmaceutical research and diagnosis (E. Place et al., Nature Materials, 8, 457-470, 2009). There is also a strong interest to use peptide-based self-assembled biomaterial such as gels for the development of molecular electronic devices (A. R. Hirst et al. Angew. Chem. Int. Ed., 47, 8002-8018, 2008).

A variety of “smart peptide hydrogels” have been generated that reaction external manipulations such as temperature, pH, mechanical influences or other stimuli with a dynamic behavior of swelling, shrinking or decomposing. Nevertheless, these biomaterials are still not “advanced” enough to mimic the biological variability of natural tissues as for example the extracellular matrix (ECM) or cartilage tissue or others. The challenge for a meaningful use of peptide hydrogels is to mimic the replacing natural tissues not only as “space filler” or mechanical scaffold, but to understand and cope with the biochemical signals and physiological requirements that keep the containing cells in the right place and under “in vivo” conditions (R. Fairman and K. Akerfeldt, Current Opinion in Structural Biology, 15, 453-463, 2005).

Much effort has been undertaken to understand and control the relationship between peptide sequence and structure for a rational design of suitable hydrogels. In general hydrogels contain macroscopic structures such as fibers that entangle and form meshes. Most of the peptide-based hydrogels utilize 0-pleated sheets which assemble to fibers as building blocks (S. Zhang et al., PNAS, 90, 3334-3338, 1993: A. Aggeli et al., Nature, 386, 259-262, 1997, etc.). It is also possible to obtain self-assembled hydrogels from α-helical peptides besides 0-sheet structure-based materials (W. A. Petka et al., Science, 281, 389-392, 1998; C. Wang et al., Nature, 397, 417-420, 1999; C. Gribbon et al., Biochemistry, 47, 10365-10371, 2008; E. Banwell et al., Nature Materials, 8, 596-600, 2009, etc.).

Nevertheless, the currently known peptide hydrogels are in most of the cases associated with low rigidity, sometimes unfavourable physiological properties and/or complexity and the requirement of substantial processing thereof which leads to high production costs. There is therefore a widely recognized need for peptide hydrogels that are easily formed, non-toxic and have a sufficiently high rigidity for standard applications. The hydrogels should also be suitable for the delivery of bioactive moieties (such as nucleic acids, small molecule therapeutics, cosmetic and anti-microbial agents) and/or for use as biomimetic scaffolds that support the in vivo and in vitro growth of cells and facilitate the regeneration of native tissue and/or for use in 2D and/or 3D biofabrication.

“Biofabrication” utilizes techniques such as additive manufacturing (i.e. printing) and moulding to create 2D and 3D structures from biomaterial building blocks. During the fabrication process, bioactive moieties and cells can be incorporated in a precise fashion. In the specific example of “bio-printing”, a computer-aided device is used to precisely deposit the biomaterial building block (ink), using a layer-by-layer approach, into the pre-determined, prescribed 3D geometry. The size of these structures range from the micro-scale to larger structures. Additives such as growth factors, cytokines, vitamins, minerals, oligonucleotides, small molecule drugs, and other bioactive moieties, and various cell types can also be accurately deposited concurrently or subsequently. Bio-inert components can be utilized as supports or fillers to create open inner spaces to mimic biological tissue. Such biological constructs can be subsequently implanted or used to investigate the interactions between cells and/or biomaterials, as well as to develop 3D disease models. In the specific example of “moulding”, the biomaterial building block is deposited into a template of specific shape and dimensions, together with relevant bioactive moieties and cells (Malda J., et al. Engineering Hydrogels for Biofabrication. Adv. Mater. (2013); Murphy S. V., et al. Evaluation of Hydrogels for Bio-printing Applications. J. of Biomed. Mater. Res. (2012)).

### SUMMARY OF THE INVENTION

It is therefore desirable to provide a biocompatible compound that is capable of forming a hydrogel, that meets at least some of the above requirements to a higher extent than currently available hydrogels and that is not restricted by the above mentioned limitations.

The objects of the present invention are solved by a hydrophobic peptide and/or peptidomimetic capable of forming a (nanofibrous) hydrogel, the hydrophobic peptide and/or peptidoinimetic having the general formula II:

Z—(X)ₐ—Z′_{b} II
wherein
Z is an N-terminal protecting group;
X is a hydrophobic amino acid sequence of aliphatic amino acids, which, at each occurrence, are independently selected from the group consisting of aliphatic amino acids and aliphatic amino acid derivatives;
a is an integer selected from 2 to 6, preferably 2 to 5;
Z′ is a C-terminal group; and
b is 0 or 1.

The inventors have found that said aliphatic amino acids and aliphatic amino acid derivatives need to exhibit an overall decrease in hydrophobicity from the N-terminus to the C-terminus of said peptide and/or peptidomimetic in order to form nanofibrous hydrogels.

The terms “peptoid” and “peptidomimetic” are used herein interchangeably and refer to molecules designed to mimic a peptide. Peptoids or peptidomimetics can arise either from modification of an existing peptide, or by designing similar systems that mimic peptides. These modifications involve changes to the peptide that will not occur naturally (such as altered backbones and/or the incorporation of non-natural amino acids).

In particular, peptoids are a subclass of peptidomimetics. In peptoids, the side chains are connected to the nitrogen of the peptide backbone, differently to normal peptides. Peptidomitnetics can have a regular peptide backbone where only the normally occurring amino acids are exchanged with a chemically different but similar amino acids, such as leucine to norleucine. In the present disclosure, the terms are used interchangeably.

In one embodiment, said aliphatic amino acids and aliphatic amino acid derivatives are either D-amino acids or L-amino acids.

In one embodiment, said aliphatic amino acids are selected from the group consisting of alanine (Ala, A), homoallylglycine, homopropargylglycine, isoleucine (Ile, I), norleucine, leucine (Leu, L), valine (Val, V) and glycine (Gly, G), preferably from the group consisting of alanine (Ala, A), isoleucine (Ile, I), leucine (Leu, L), valine (Val, V) and glycine (Gly, G).

In one embodiment, all or a portion of said aliphatic amino acids are arranged in an order of decreasing amino acid size in the direction from N- to C-terminus, wherein the size of the aliphatic amino acids is defined as I=L>V>A>G.

In one embodiment, said aliphatic amino acids arranged in an order of decreasing amino acid size have a sequence which is a non-repetitive sequence.

In one embodiment, the very first N-terminal amino acid of said aliphatic amino acids is less crucial (it can be G, V or A). The inventors found that this specific first amino acid has not a dominant on this otherwise mandatory requirement of decreasing hydrophobicity from N- to C-terminus.

In one embodiment, the first N-terminal amino acid of said aliphatic amino acids is G, V or A.

In one embodiment, said aliphatic amino acids have a sequence selected from

(SEQ ID NO: 1)
ILVAG
(SEQ ID NO: 2)
LIVAG,
(SEQ ID NO: 3)
IVAG,
(SEQ ID NO: 4)
LVAG,
(SEQ ID NO: 5)
ILVA,
(SEQ ID NO: 6)
LIVA,
(SEQ ID NO: 13)
IVG,
(SEQ ID NO: 14)
VIG,
(SEQ ID NO: 15)
IVA,
(SEQ ID NO: 16)
VIA,
(SEQ ID NO: 17)
VI
and
(SEQ ID NO: 18)
IV,

wherein, optionally, there is an G, V or A preceding such sequence at the N-terminus, such as

(SEQ ID NO. 7)
AIVAG,
(SEQ ID NO. 8)
GIVAG,
(SEQ ID NO. 9)
VIVAG,
(SEQ ID NO. 10)
ALVAG,
(SEQ ID NO. 11)
GLVAG,
(SEQ ID NO. 12)
VLVAG.

In one embodiment, (X)ₐ has a sequence selected from the group consisting of SEQ ID NOs. 1 to 18,

preferably the sequence with SEQ ID NO: 1 and SEQ ID NO: 2.

In one embodiment, all or a portion of the aliphatic amino acids are arranged in an order of identical amino acid size, preferably wherein said aliphatic amino acids arranged in order of identical amino acid size have a sequence with a length of 2 to 4 amino acids.

For example, said aliphatic amino acids arranged in an order of identical size have a sequence selected from LLLL, LLL, LL, IIII, III, II, VVVV, VVV, VV, AAAA, AAA, AA, GGGG, GGG, and GG.

In one embodiment, said N-terminal protecting group Z has the general formula —C(O)—R,

wherein R is selected from the group consisting of H, unsubstituted or substituted alkyls, and unsubstituted or substituted aryls,
wherein R is preferably selected from the group consisting of methyl, ethyl, propyl, isopropyl, butyl and isobutyl.

In one embodiment, said N-terminal protecting group Z is an acetyl group.

In one embodiment, said N-terminal protecting group Z is a peptidomimetic molecule, including natural and synthetic amino acid derivatives, wherein the N-terminus of said peptidomimetic molecule may be modified with a functional group selected from the group consisting of carboxylic acid, amide, alcohol, aldehyde, amine, imine, nitrile, an urea analog, phosphate, carbonate, sulfate, nitrate, maleimide, vinyl sulfone, azide, alkyne, alkene, carbohydrate, imide, peroxide, ester, aryl, ketone, sulphite, nitrite, phosphonate, and silane.

In one embodiment, said C-terminal group Z′ is a non-amino acid, preferably selected from the group of small molecules, functional groups and linkers. Such C-terminal groups Z′ can be polar or non-polar moieties used to functionalize the peptide and/or peptidomimetic of the invention.

In one embodiment, said C-terminal group Z′ is selected from
functional groups, such as polar or non-polar functional groups,
   such as (but not limited to)
      —COOH, —COOR, —COR, —CONHR or —CONRR′ with R and R′ being selected from the group consisting of H, unsubstituted or substituted alkyls, and unsubstituted or substituted aryls,
      —NH₂, —OH, —SH, —CHO, maleimide, imidoester, carbodiimide ester, isocyanate;
small molecules,
   such as (but not limited to) sugars, alcohols, hydroxy acids, amino acids, vitamins, biotin;
linkers terminating in a polar functional group,
   such as (but not limited to) ethylenediamine, PEG, carbodiimide ester, imidoester;
linkers coupled to small molecules or vitamins,
   such as biotin, sugars, hydroxy acids,

In one embodiment, wherein said C-terminal group Z′ can be used for chemical conjugation or coupling of at least one compound selected from
bioactive molecules or moieties,
   such as growth factors, cytokines, lipids, cell receptor ligands, hormones, prodrugs, drugs, vitamins, antigens, antibodies, antibody fragments, oligonucleotides (including but not limited to DNA, messenger RNA, short hairpin RNA, small interfering RNA, microRNA, peptide nucleic acids, aptamers), saccharides;
label(s), dye(s),
   such as fluorescent or radioactive label(s), imaging contrast agents;
pathogens,
   such as viruses, bacteria and parasites;
micro- and nanoparticles
or combinations thereof
   wherein said chemical conjugation can be carried out before or after self-assembly of the peptide and/or peptidomimetic.

In one embodiment, the C-terminus of the peptide and/or peptidomimetic is functionalized (without the use of a C-terminal group or linker), such as by chemical conjugation or coupling of at least one compound selected from
bioactive molecules or moieties,
   such as growth factors, cytokines, lipids, cell receptor ligands, hormones, prodrugs, drugs, vitamins, antigens, antibodies, antibody fragments, oligonucleotides (including but not limited to DNA, messenger RNA, short hairpin RNA, small interfering RNA, microRNA, peptide nucleic acids, aptamers), saccharides;
label(s), dye(s),
   such as fluorescent or radioactive label(s), imaging contrast agents;
pathogens,
   such as viruses, bacteria and parasites;
micro- and nanoparticles
or combinations thereof
   wherein said chemical conjugation can be carried out before or after self-assembly of the peptide and/or peptidomimetic.

In one embodiment, said C-terminal group Z′ is a peptidomimetic molecule, including natural and synthetic amino acid derivatives, wherein the C-terminus of said peptidomimetic molecule may be modified with a functional group selected from the group consisting of carboxylic acid, amide, alcohol, aldehyde, amine, imine, nitrile, an urea analog, phosphate, carbonate, sulfate, nitrate, maleimide, vinyl sulfone, azide, alkyne, alkene, carbohydrate, imide, peroxide, ester, aryl, ketone, sulphite, nitrite, phosphonate, and silane.

In one embodiment, the hydrophobic peptide and/or peptidomimetic according to the invention is being stable in aqueous solution at physiological conditions at ambient temperature for a period of time in the range from 1 day to at least 6 months, preferably to at least 8 months more preferably to at least 12 months.

In one embodiment, the hydrophobic peptide and/or peptidomimetic according to the invention is being stable in aqueous solution at physiological conditions, at a temperature up to 90° C., for at least 1 hour.

The objects of the present invention are solved by a composition or mixture comprising

(a) at least one hydrophobic peptide and/or peptidomimetic of the present invention, and
(b) at least one hydrophobic peptide and/or peptidomimetic capable of forming a hydrogel, the hydrophobic peptide and/or peptidomimetic having the general formula:

Z—(X)ₐ—N′_{b}
wherein
Z is as defined herein for the hydrophobic peptide and/or peptidomimetic of the present invention;
X is as defined herein for the hydrophobic peptide and/or peptidomimetic of the present invention;
a is as defined herein for the hydrophobic peptide and/or peptidomimetic of the present invention;
N′ is a non-polar C-terminal group which differs from Z′, the polar C-terminal group as defined herein for the hydrophobic peptide and/or peptidomimetic of the present invention;
   and is preferably carboxylic acid, amide, alcohol, biotin, inaleimide, sugars, and hydroxyacids,
and
b is 0 or 1.

The objects of the present invention are solved by a hydrogel comprising the hydrophobic peptide and/or peptidomimetic of the present invention.

In one embodiment, the hydrogel is stable in aqueous solution at ambient temperature for a period of at least 7 days, preferably at least 2 to 4 weeks, more preferably at least 1 to 6 months.

In one embodiment, the hydrogel is characterized by a storage modulus G′ to loss modulus G″ ratio that is greater than 2.

In one embodiment, the hydrogel is characterized by a storage modulus G′ from 100 Pa to 80,000 Pa at a frequency in the range of from 0.02 Hz to 16 Hz.

In one embodiment, the hydrogel has a higher mechanical strength than collagen or its hydrolyzed form (gelatin).

The objects of the present invention are solved by a hydrogel comprising

(a) at least one hydrophobic peptide and/or peptidomimetic of the present invention, and
(b) at least one hydrophobic peptide and/or peptidomimetic with a non-polar head group.

Said at least one “hydrophobic peptide and/or peptidomimetic with a non-polar head group” is capable of forming a hydrogel and has the general formula:

Z—(X)ₐ—N′_{b}
wherein
Z, X and a are as defined herein for the hydrophobic peptide and/or peptidomimetic of the present invention;
N′ is a non-polar C-terminal group which differs from Z′, the polar C-terminal group as defined herein for the hydrophobic peptide and/or peptidomimetic of the present invention;
   and is preferably carboxylic acid, amide, alcohol, biotin, maleimide, sugars, and hydroxyacids,
and
b is 0 or 1.

In one embodiment, the hydrogel comprises fibers of the hydrophobic peptide and/or peptidomimetic of the invention or fibers of the hydrophobic peptide and/or peptidomimetic with a non-polar head group as defined above, said fibers defining a network that is capable of entrapping at least one of a microorganism, a virus particle, a peptide, a peptoid, a protein, a nucleic acid, an oligosaccharide, a polysaccharide, a vitamin, an inorganic molecule, a synthetic polymer, a small organic molecule, a micro- or nanoparticle or a pharmaceutically active compound.

In one embodiment, the hydrogel comprises at least one of a microorganism, a virus particle, a peptide, a peptoid, a protein, a nucleic acid, an oligosaccharide, a polysaccharide, a vitamin, an inorganic molecule, a synthetic polymer, a small organic molecule, a micro- or nanoparticle or a pharmaceutically active compound entrapped by the network of fibers of the hydrophobic polymer.

In one embodiment, the fibers of the hydrophobic polymer are coupled to the at least one of a microorganism, a virus particle, a peptide, a peptoid, a protein, a nucleic acid, an oligosaccharide, a polysaccharide, a vitamin, an inorganic molecule, a synthetic polymer, a small organic molecule, a micro- or nanoparticle or a pharmaceutically active compound entrapped by the network of fibers of the amphiphilic polymer.

In one embodiment, the hydrogel is comprised in at least one of a fuel cell, a solar cell, an electronic cell, a biosensing device, a medical device, an implant, a pharmaceutical composition and a cosmetic composition.

In one embodiment, the hydrogel is injectable.

The objects of the present invention are solved by the use of the hydrogel according to the present invention in at least one of the following:
release of a pharmaceutically active compound and/or delivery of bioactive moieties,
medical tool kit,
a fuel cell,
a solar cell,
an electronic cell,
regenerative medicine and tissue regeneration,
wound healing,
2D and 3D synthetic cell culture substrate,
stem cell therapy,
injectable therapies,
biosensor development,
biofunctionalized surfaces,
biofabrication, such as bio-printing, and
gene therapy.

For the uses, we also refer to the uses in biofabrication described in the inventors' parallel application “Self-assembling peptides, peptidomimetics and peptidic conjugates as building blocks for biofabrication and printing”, having the same filing date as the present application, and the subsequent embodiments and methods described therein, which also apply to the hydrophobic peptides and/or peptidomimetics of this invention.

The objects of the present invention are solved by a method of preparing a hydrogel, the method comprising dissolving a hydrophobic peptide and/or peptidomimetic according to the present invention in an aqueous solution.

In one embodiment, the dissolved hydrophobic peptide and/or peptidomimetic in aqueous solution is further exposed to temperature, wherein the temperature is in the range from 20° C. to 90° C., preferably from 20° C. to 70° C.

In one embodiment, the hydrophobic peptide and/or peptidomimetic is dissolved at a concentration from 0.01 μg/ml to 100 mg/ml, preferably at a concentration from 1 mg/ml to 50 mg/ml, more preferably at a concentration from about 1 mg/ml to about 20 mg/ml.

The objects of the present invention are solved by a method of preparing a hydrogel, the method comprising dissolving a hydrophobic peptide and/or peptidomimetic according to the present invention and a hydrophobic peptide and/or peptidomimetic with a non-polar head group as defined herein in an aqueous solution.

The objects of the present invention are solved by a wound dressing or wound healing agent comprising a hydrogel according to the invention.

The objects of the present invention are solved by a surgical implant, or stent, the surgical implant or stent comprising a peptide and/or peptidomimetic scaffold, wherein the peptide and/or peptidomimetic scaffold is formed by a hydrogel according to the invention.

The objects of the present invention are solved by a pharmaceutical and/or cosmetic composition and/or a biomedical device and/or electronic device comprising the hydrophobic peptide and/or peptidomimetic according to the invention.

The objects of the present invention are solved by a pharmaceutical and/or cosmetic composition and/or a biomedical device and/or electronic device comprising the hydrophobic peptide and/or peptidomimetic of the present invention and the hydrophobic peptide and/or peptidomimetic with a non-polar head group as defined herein.

In one embodiment, the pharmaceutical and/or cosmetic composition and/or the biomedical device, and/or the electronic devices further comprises a pharmaceutically active compound.

In one embodiment, the pharmaceutical and/or cosmetic composition is provided in the form of a topical gel or cream, a spray, a powder, or a sheet, patch or membrane, or wherein the pharmaceutical and/or cosmetic composition is provided in the form of an injectable solution.

In one embodiment, the pharmaceutical and/or cosmetic composition further comprises a pharmaceutically acceptable carrier.

The objects of the present invention are solved by a kit of parts, the kit comprising a first container with a hydrophobic peptide and/or peptidomimetic according to the invention and a second container with an aqueous solution.

In one embodiment, the kit further comprises a third container with a hydrophobic peptide and/or peptidomimetic with a non-polar head group as defined herein.

In one embodiment, the aqueous solution of the second container further comprises a pharmaceutically active compound.

and/or wherein the first and/or third container with a hydrophobic peptide and/or peptidomimetic further comprises a pharmaceutically active compound.

The objects of the present invention are solved by an in vitro or in vivo method of tissue regeneration comprising the steps:
(a) providing a hydrogel according to the invention,
(b) exposing said hydrogel to cells which are to fonn regenerated tissue,
(c) allowing said cells to grow on said hydrogel.

In one embodiment, wherein the method is performed in vivo, in step a), said hydrogel is provided at a place in a body where tissue regeneration is intended,

wherein said step a) is preferably performed by injecting said hydrogel at a place in the body where tissue regeneration is intended.

The objects of the present invention are solved by a method of treatment of a wound and for wound healing, said method comprising the step of
applying an effective amount of a hydrogel according to the invention or a pharmaceutical composition according to the invention to a wound.

The objects of the present invention are solved by a bioimaging device comprising a hydrogel according to the invention for in vitro and/or in vivo use,

preferably for oral application, for injection and/or for topical application.

The objects of the present invention are solved by a 2D or 3D cell culture substrate comprising a hydrogel according to the invention.

The peptides, peptidomimetics and peptoids disclosed herein are suitable as ink(s) or (biomaterial) building block(s) in biofabrication, including bioprinting, (bio)moulding.

“Biofabrication” as used herein refers to the use of techniques, such as additive manufacturing (i.e. bio-printing) and moulding to create 2D and 3D structures or biological constructs from biomaterial building blocks (i.e. the peptides and/or peptoids according to the invention). During the fabrication process, bioactive moieties and cells can be incorporated in a precise fashion. In the specific example of “bio-printing”, a computer-aided device is used to precisely deposit the biomaterial building block (ink), using a layer-by-layer approach, into the pre-determined, prescribed 3D geometry. The size of these structures range from the micro-scale to larger structures. Additives such as growth factors, cytokines, vitamins, minerals, oligonucleotides, small molecule drugs, and other bioactive moieties, and various cell types can also be accurately deposited concurrently or subsequently. Bio-inert components can be utilized as supports or fillers to create open inner spaces to mimic biological tissue. Such biological constructs can be subsequently implanted or used to investigate the interactions between cells and/or biomaterials, as well as to develop 3D disease models. In the specific example of “moulding”, the biomaterial building block is deposited into a template of specific shape and dimensions, together with relevant bioactive moieties and cells.

(see Malda J., et al. Engineering Hydrogels for Biofabrication. Adv. Mater. (2013); Murphy S. V., et al. Evaluation of Hydrogels for Bio-printing Applications. J. of Biomed. Mater. Res. (2012)).

“Bioprinting” is part of the field tissue engineering which is the use of a combination of cells, engineering and materials methods, and suitable biochemical and physio-chemical factors to improve or replace biological functions.

Tissue engineering is used to repair or replace portions of or whole tissues (i.e., bone, cartilage, blood vessels, bladder, skin, muscle etc.). Often, the tissues involved require certain mechanical and structural properties for proper functioning.

The term “bioprinting” as used herein also comprises a process of making a tissue analog by depositing scaffolding or ink material (the peptides/peptoids of the invention or hydrogels thereof) alone, or mixed with cells, based on computer driven mimicking of a texture and a structure of a naturally occurring tissue.

An “ink” or “bio-ink” for bioprinting as used herein refers to the biomaterial building block that is sequentially deposited to build a macromolecular scaffold.

In one embodiment, the C-terminal amino acid is further functionalized.

In one embodiment, the polar functional group(s) can be used for chemical conjugation or coupling of at least one compound selected from
bioactive molecules or moieties,
   such as growth factors, cytokines, lipids, cell receptor ligands, hormones, prodrugs, drugs, vitamins, antigens, antibodies, antibody fragments, oligonucleotides (including but not limited to DNA, messenger RNA, short hairpin RNA, small interfering RNA, microRNA, peptide nucleic acids, aptamers), saccharides;
label(s), dye(s),
   such as imaging contrast agents;
pathogens,
   such as viruses, bacteria and parasites;
micro- and nanoparticles
or combinations thereof
   wherein said chemical conjugation can be carried out before or after self-assembly of the peptide and/or peptoid.

In one embodiment, the use according to the invention comprises a conformational change of the peptide(s) and/or peptoid(s) during self-assembly,

preferably a conformational change from a random coil conformation to a helical intermediate structure (such as α-helical fibrils) to a final beta turn or cross beta conformation, such as fibrils which further aggregate and/or condense into nanofibers (which make up a network), wherein, preferably, the conformational change is dependent on the peptide concentration, ionic environment, pH and temperature.

In one embodiment, at least one peptide and/or peptoid as herein defined forms a hydrogel.

The hydrogel is formed by self-assembly of the peptide and/or peptiod, as explained in further detail below.

In one embodiment, different peptide(s) and/or peptoid(s) as defined herein are used to form the hydrogel.

Preferably, different peptide(s) and/or peptoid(s) refers to peptide(s) and/or peptoid(s) that differ in their amino acid sequence, C-terminal group(s), conjugated/coupled compounds (such as different labels, bioactive molecules etc) or combinations thereof.

In one embodiment, at least one peptide and/or peptoid as defined herein is dissolved in water and wherein the solution obtained can be dispensed through needles and print heads.

In one embodiment, the use according to the invention comprises conjugation or coupling of further compound(s) to the peptides and/or peptoid, preferably to C-terminal group(s), post-assembly,
wherein said further compound(s) can be selected from
   bioactive molecules or moieties,
      such as growth factors, cytokines, lipids, cell receptor ligands, hormones, prodrugs, drugs, vitamins, antigens, antibodies, antibody fragments, oligonucleotides (including but not limited to DNA, messenger RNA, short hairpin RNA, small interfering RNA, microRNA, peptide nucleic acids, aptamers), saccharides;
   label(s), dye(s),
      such as imaging contrast agents;
   pathogens,
      such as viruses, bacteria and parasites;
   micro- and nanoparticles
   or combinations thereof.

In one embodiment, the peptide and/or peptoid is present at a concentration in the range of from 0.1% to 30% (w/w), preferably 0.1% to 20% (w/w), more preferably 0.1% to 10% (w/w), more preferably 0.1% to 5% (w/w), even more preferably 0.1% to 3% (w/w), with respect to the total weight of said hydrogel.

In one embodiment, the use according to the invention comprises the addition or mixing of cells prior or during gelation, which are encapsulated by the hydrogel,
wherein said cells can be stem cells (mesenchymal, progenitor, embryonic and induced pluripotent stem cells), transdifferentiated progenitor cells and primary cells isolated from patient samples (fibroblasts, nucleus pulposus).
   preferably comprising the addition of further compound(s) prior or during gelation, which are co-encapsulated by the hydrogel.

In one embodiment, the use according to the invention comprises the addition of cells onto the printed hydrogel, wherein said cells can be stem cells (adult, progenitor, embryonic and induced pluripotent stein cells), transdifferentiated progenitor cells, and primary cells (isolated from patients) and cell lines (such as epithelial, neuronal, hematopoietic and cancer cells).

In one embodiment, the use according to the invention comprises

(1) the addition or mixing of cells prior or during gelation, which are encapsulated by the hydrogel, and
(2) subsequently comprising the addition of cells onto the printed hydrogel, wherein said cells of (1) and (2) are the same or different,
and can be stem cells (adult, progenitor, embryonic and induced pluripotent stein cells), transdifferentiated progenitor cells, and primary cells (isolated from patients) and cell lines (such as epithelial, neuronal, hematopoietic and cancer cells).

In one embodiment, the use according to the invention comprises the addition of cross-linkers to the peptide(s) and/or peptoid(s),

wherein said cross-linkers preferably include short linkers, linear and branched polymers, polymers conjugated with bioactive molecules or moieties.

The objects of the present invention are solved by a method of preparing a hydrogel, the method comprising dissolving at least one peptide and/or peptoid as defined herein in an aqueous solution, such as water, or in a polar solvent, such as ethanol.

In one embodiment, the method of the invention comprises stimuli-responsive gelation of the at least one peptide and/or peptoid as defined herein,

wherein said stimulus/stimuli or gelation condition(s) is/are selected from pH, salt concentration and/or temperature.

In one embodiment, the at least one peptide and/or peptoid comprises as the polar head group basic amino acid(s), such as lysine or lysine-mimetic molecules, preferably ainidated basic amino acid(s),

and gelation is carried out in the presence of salt at physiological conditions (such as PBS or 0.9% saline and PBS) and/or at a pH above physiological pH, preferably pH 7 to 10 (such as by adding NaOH).

In one embodiment, the at least one peptide and/or peptoid comprises as the polar head group acidic amino acid(s),

and gelation is carried out at a pH below physiological pH 7, preferably pH 2 to 6.

In one embodiment, the dissolved peptide and/or peptoid is further warmed or heated, wherein the temperature is in the range from 20° C. to 90° C., preferably from about 30° C. to 70° C., more preferably from about 37° C. to 70° C.

In one embodiment, the at least one peptide and/or peptoid is dissolved at a concentration from 0.01 μg/ml to 100 mg/ml, preferably at a concentration from 1 mg/ml to 50 mg/ml, more preferably at a concentration from about 1 mg/ml to about 20 mg/ml.

The objects of the present invention are solved by a method of preparing continuous fibres, the method comprising
dissolving at least one peptide and/or peptoid as defined herein in an aqueous solution, such as water, and
dispensing the solution obtained through needles, print heads, fine tubings and/or microfluidic devices into a buffered solution, such as PBS.

In one embodiment, the method comprises the addition of further compound(s) prior or during gelation/self-assembly, which are encapsulated by the hydrogel,
wherein said further compound(s) can be selected from
   bioactive molecules or moieties,
      such as growth factors, cytokines, lipids, cell receptor ligands, hormones, prodrugs, drugs, vitamins, antigens, antibodies, antibody fragments, oligonucleotides (including but not limited to DNA, messenger RNA, short hairpin RNA, small interfering RNA, microRNA, peptide nucleic acids, aptamers), saccharides;
   label(s), dye(s),
      such as imaging contrast agents;
   pathogens,
      such as viruses, bacteria and parasites;
   quantum dots, nano- and microparticles,
   or combinations thereof.

In one embodiment, the method comprises the addition or mixing of cells prior or during gelation/self-assembly, which are encapsulated by the hydrogel,
wherein said cells can be stein cells (mesenchymal, progenitor, embryonic and induced pluripotent stem cells), transdifferentiated progenitor cells and primary cells isolated from patient samples (fibroblasts, nucleus pulposus).
   preferably comprising the addition of further compound(s) prior or during gelation (such as defined herein), which are co-encapsulated by the hydrogel.

In one embodiment, the method comprises the addition of cells onto the printed hydrogel, wherein said cells can be stem cells (adult, progenitor, embryonic and induced pluripotent stem cells), transdifferentiated progenitor cells, and primary cells (isolated from patients) and cell lines (such as epithelial, neuronal, hematopoietic and cancer cells).

In one embodiment, the method comprises the following steps:

(1) the addition or mixing of cells prior or during gelation, which are encapsulated by the hydrogel, and
(2) subsequently the addition of cells onto the printed hydrogel,
wherein said cells of (1) and (2) are the same or different,
and can be stem cells (adult, progenitor, embryonic and induced pluripotent stem cells), transdifferentiated progenitor cells, and primary cells (isolated from patients) and cell lines (such as epithelial, neuronal, hematopoietic and cancer cells).

In one embodiment, the method comprises the addition of cross-linkers to the peptide(s) and/or peptoid(s) prior, during or after gelation/self-assembly,

wherein said cross-linkers preferably include short linkers, linear and branched polymers, polymers conjugated with bioactive molecules or moieties (such as defined herein), wherein, preferably, said cross-linkers interact electrostatically with the peptides and/or peptoid(s) during self-assembly.

In one embodiment, the method comprises the use of different peptide(s) and/or peptoid(s).

Preferably, different peptide(s) and/or peptoid(s) refers to peptide(s) and/or peptoid(s) that differ in their amino acid sequence, C-terminal group(s), conjugated/coupled compounds (such as different labels, bioactive molecules etc) or combinations thereof.

The objects of the present invention are solved by the use of a hydrogel obtained by a method (for preparing a hydrogel and/or for preparing continuous fibers) according to the invention for substrate-mediated gene delivery,

wherein oligonucleotides are encapsulated in the hydrogel and cells are co-encapsulated or seeded onto said hydrogel.

The objects of the present invention are solved by the use (of a peptide and/or peptoid for biofabrication) according to the invention or the use of a hydrogel obtained by a method (for preparing a hydrogel and/or for preparing continuous fibers) according to the invention, for obtaining 2D mini-hydrogel arrays,

preferably comprising using printers, pintools and micro-contact printing.

Preferably, a microarray of the invention comprises hydrogels that encapsulate different biomolecules, drugs, compounds, cells etc.

In one embodiment, said use comprises printing the 2D mini-hydrogels onto electrical circuits or piezoelectric surfaces that conduct current.

The objects of the present invention are solved by the use (of a peptide and/or peptidomimetic for biofabrication) according to the invention or the use of a hydrogel obtained by a method (for preparing a hydrogel and/or for preparing continuous fibers) according to the invention, as injectable or for injectable therapies,

such as for the treatment of degenerative disc disease.

An injectable is preferably an injectable scaffold or an injectable implant or an implantable scaffold.

By virtue of their self-assembling properties, the stimuli-responsive ultrashort peptides of the present invention are ideal candidates for injectable scaffolds. Such scaffolds can be injected as semi-viscous solutions that complete assembly in situ. Irregular-shaped defects can be fully filled, facilitating scaffold integration with native tissue. These injectable formulations offer significant advantages over ex vivo techniques of preparing nanofibrous scaffolds, such as electrospinning, which have to be surgically implanted. During the process of in situ gelation, the ability to modulate gelation rate enables the clinician to sculpt the hydrogel construct into the desired shape for applications such as dermal fillers. Furthermore, the bio compatibility and in vivo stability bodes well for implants that need to persist for several months. Taking into consideration the stiffness and tunable mechanical properties, we are particularly interested in developing injectable therapies and implantable scaffolds that fulfill mechanically supportive roles.

The objects of the present invention are solved by the use (of a peptide and/or peptoid for biofabrication) according to the invention or the use of a hydrogel obtained by a method (for preparing a hydrogel and/or for preparing continuous fibers) according to the invention, comprising bioprinting, such as 3D microdroplet printing, and biomoulding.

In one embodiment, said use is for obtaining 3D organoid structures or 3D macromolecular biological constructs.

An organoid structure is a structure resembling an organe.

The term “3D organoid structures” or “3D macromolecular biological constructs” refers to samples in which various cell types are integrated in a 3D scaffold containing various biochemical cues, in a fashion which resembles native tissue. These constructs can potentially be used as implants, disease models and models to study cell-cell and cell-substrate interactions.

In one embodiment, said use comprises the use of moulds (such as of siliconde) to pattern the hydrogels in 3D.

In one embodiment, said use is for obtaining multi-cellular constructs,

which comprise different cells/cell types,
which preferably comprise co-encapsulated further compound(s) (such as defined herein) and/or cross-linkers (such as defined herein).

In one embodiment, said use is for obtaining 3D cellular constructs or scaffolds comprising encapsulated cells and cells deposited or printed onto the surface of the printed/fabricated scaffold.

In one embodiment, said use is for
preparation of cell based assays,
   preferably for identifying patient specimens, more preferably for identifying patient specimens containing pathogens (e.g. dengue, malaria, norovirus), which do not infect primary cells that have lost their native phenotype;
recovery of infected cells to identify and expand pathogen(s) of interest,
   preferably for elucidating mechanism(s) of infection and/or enabling the design of molecules that inhibit pathogen infection and/or replication.

The objects of the present invention are solved by a method for obtaining a multi-cellular construct, comprising
preparing a hydrogel by the method (for preparing a hydrogel and/or for preparing continuous fibers) according to the invention,
comprising the addition or mixing of different cells or cell types prior or during
gelation/self-assembly, which are encapsulated by the hydrogel,
   wherein said cells can be stem cells (mesenchymal, progenitor, embryonic and induced pluripotent stem cells), transdifferentiated progenitor cells and primary cells isolated from patient samples (fibroblasts, nucleus pulposus).
preferably comprising the addition of further compound(s) (such as defined herein) prior or during gelation, which are co-encapsulated by the hydrogel,
optionally comprising the addition of cross-linkers (such as defined herein) to the peptide(s) and/or peptoid(s) prior or during gelation/self-assembly,
obtaining the multi-cellular construct.

The objects of the present invention are solved by a method for obtaining a multi-cellular construct, comprising
preparing a hydrogel by the method (for preparing a hydrogel and/or for preparing continuous fibers) according to the invention,
comprising the following steps:
(1) the addition or mixing of cells prior or during gelation, which are encapsulated by the hydrogel, and
(2) subsequently the addition of cells onto the printed hydrogel,
wherein said cells of (1) and (2) are different,
and can be stem cells (adult, progenitor, embryonic and induced pluripotent stein cells), transdifferentiated progenitor cells, and primary cells (isolated from patients) and cell lines (such as epithelial, neuronal, hernatopoietic and cancer cells),
preferably comprising the addition of further compound(s) (such as defined herein) prior or during gelation, which are co-encapsulated by the hydrogel,
optionally comprising the addition of cross-linkers (such as defined herein) to the peptide(s) and/or peptidomimetic(s) prior or during gelation/self-assembly,
obtaining the multi-cellular construct.

In one embodiment, the multi-cellular construct obtained is formed in a mould (such as of silicone).

The objects of the present invention are solved by a multi-cellular construct obtained according to the methods for obtaining a multi-cellular construct according to the invention and as described herein above,
preferably comprising micro-domains.

The objects of the present invention are solved by the use of a 3D biological construct obtained by a method (for obtaining a 3D biological construct) according to the invention or of a multi-cellular construct obtained according to the method (for obtaining a multi-cellular construct) according to the invention as:
organoid model for screening biomolecule libraries, studying cell behavior, infectivity of pathogens and disease progression, screening infected patient samples, evaluating drug efficacy and toxicity,
tissue-engineered implant for regenerative medicine, and/or
in vitro disease model.

In one embodiment, said use is for
preparation of cell based assays,
   preferably for identifying patient specimens, more preferably for identifying patient specimens containing pathogens (e.g. dengue, malaria, norovirus), which do not infect primary cells that have lost their native phenotype;
recovery of infected cells to identify and expand pathogen(s) of interest,
   preferably for elucidating mechanism(s) of infection and/or enabling the design of molecules that inhibit pathogen infection and/or replication.

### BRIEF DESCRIPTION OF THE DRAWINGS

Reference is now made to the figures, wherein:

FIG. 1. Self-assembly of ultrashort peptides/peptidomimetics into macromolecular nanofibrous hydrogels.

(A) These amphiphilic peptides have the characteristic motif, wherein the aliphatic amino acids are arranged in decreasing hydrophobicity from N-terminus. During self-assembly, the peptides are hypothesized to associate in an anti-parallel fashion, giving rise to α-helical intermediate structures detected by circular dichroism. (B) As the peptide concentration increases, conformational changes from random coil (black line) to α-helical intermediates (red line) to β-fibrils (blue line) are observed. The insert better illustrates the latter conformations. This phenomenon is observed for hexamers and trimers, though the transition concentration to β-fibrils is higher for the trimer. The peptide dimers subsequently stack in fibrils that aggregate into nanofibers and sheets, which entrap water to form hydrogels. (C) The nanofibrous architecture, as observed using field emission scanning microscopy, resembles extracellular matrix. The fibers extend into the millimeter range. The nanofibers of hexamers readily condense into sheets, while individual fibers are more easily observed for trimers. The fibers form interconnected three-dimensional scaffolds which are porous.

FIG. 2. Examples of subclasses of peptides/peptidomimetics that demonstrate stimuli-responsive gelation. (refers to the inventors' parallel application “Self-assembling peptides, peptidomimetics and peptidic conjugates as building blocks for biofabrication and printing”, having the same filing date as the present application)

FIG. 3. Stimuli-responsive gelation of amidated peptides/peptidomimetics containing primary amine groups. (refers to the inventors' parallel application “Self-assembling peptides, peptidomimetics and peptidic conjugates as building blocks for biofabrication and printing”, having the same filing date as the present application)

(A) A subclass of ultrashort peptides with lysine as the polar residue at the C-terminus, form hydrogels more readily in salt solutions—the minimum gelation concentration is significantly lowered and the gelation kinetics are accelerated. Ac-LIVAGK-NH₂ forms hydrogels at 20 mg/mL in water, 12 mg/mL in saline, 7.5 ing/mL in PBS, and 10 mg/mL in 10 mM NaOH. (B) The rigidity, as represented by the storage modulus (G′), of 20 mg/mL Ac-LIVAGK-NH₂ hydrogels increases by one order of magnitude to 10 kPa when dissolved in normal saline (NaCl) as compared to water at 1 kPa. In phosphate buffered saline (PBS), G′ increases to 40 kPa. The stiffness also increases with peptide concentration. (C) The addition of sodium hydroxide (NaOH) enhances the rigidity of 20 mg/mL Ac-LIVAGK-NH₂ hydrogel from 1 kPa in water to 80 kPa. The rigidity increases with NaOH concentration. (D) Hydrogel droplet arrays of various dimensions can be obtained by mixing equivolumes of peptide solution (such as 10 mg/mL Ac-ILNAGK-NH₂) and PBS containing small molecules. Bioactive moieties can also be encapsulated; 1 μL droplets with green food colouring and 488 nm emission quantum dots, 2 μL droplets with red food colouring and 568 nm emission fluorophore conjugated to a secondary antibody, and 5 μL droplets with methylene blue and DAPI. (E) Hydrogel “noodles” are obtained by extruding 5 mg/mL peptide solution through a 27 gauge needle into a concentrated salt bath.

FIG. 4. Cells can be encapsulated and immobilized within the peptide hydrogels for various applications such as induction of differentiation and screening assays.

(A) Human mesenchymal stem cells encapsulated within 2 μL droplets of 5 mg/mL peptide hydrogels. (Ai) Photograph of mini-hydrogels on a 25 mm cover slip. (Aii) The cells encapsulated visualised using fluorescent microscopy of a single mini-hydrogel, wherein the cells are stained with Phalliodin-FITC (cytoskeleton is stained green) and Dapi (nuclei stained blue). (Aiii) The encapsulated cells adopt an elongated morphology as demonstrated in this 2D projection image at 10× magnification. The cells are located on different focal planes. (Aiv) Higher magnification image (63×) showing the focal adhesions (in red). (B) Human mesenchymal stem cells cultured on hydrogel films also adopt an elongated morphology compared to those cultured on (C) glass cover slips.

FIG. 5. Oligonucleotides such as DNA, mRNA, siRNA can be encapsulated in the hydrogels for substrate mediated gene delivery. Cells can subsequently be co-encapsulated or seeded onto these hydrogels.

(A) Hydrogels protect the oligonucleotide from nuclease degradation. (B) Hydrogels slowly release the encapsulated DNA over time. (C) Cells cultured on hydrogels encapsulating GFP mRNA express the protein of interest (GFP) after 2 days.

FIG. 6. 2D mini-hydrogel arrays for various applications.

Such 2D arrays can be generated using existing technology such as printers, pintools and Micro-contact printing. (A) The array could be subject to electrical or magnetic stimuli, such as a electric field or point stimuli. The mini-hydrogels can also be printed onto electrical circuits or piezoelectric surfaces to conduct current. (B) Different small molecules or oligonucleotides can be encapsulated to create a biochemical gradient. (C) Different cells can be encapsulated in different mini-hydrogels and treated with the same drug/bioactive molecule dissolved in the bulk media. Alternatively, different drugs or biochemical cues can be incorporated to alter gene expression of the encapsulated cells.

FIG. 7. The stability and mechanical properties of mini-hydrogels can also be further enhanced through the addition of cross-linkers, including short linkers, linear and branched polymers.

Such composite polymer-peptide hydrogels are produced by incorporating (A) linear and (B) branched polymers that can interact electrostatically with ultrashort peptides during self-assembly. The resulting hydrogels have better mechanical properties (due to cross-linking and increased elasticity) and (C) offer opportunities to incorporate bioactive functionalities to modulate the immune and physiological response.

FIG. 8. 3D bio printingor moulding techniques to create biological constructs with distinct, multi-functional micro-niches.

Multi-cellular constructs can also be obtained as the hydrogel can spatially confine different cell types.

FIG. 9. A novel class of hydrophobic peptides which self-assemble into hydrogels.

(A) These hydrophobic peptides have the characteristic motif, wherein the aliphatic amino acids are arranged in decreasing hydrophobicity from N-terminus, as exemplified by Ac-ILVAG. (B) A hydrogel comprising of peptide Ac-ILVAG (at 5 mg/mL), which has a carboxylic acid as a polar functional group at the C-terminus.

FIG. 10. C-terminus functionalization of the hydrophobic peptides.

(A) The characteristic peptidic motif that drives self-assembly can be coupled to other functional groups, linkers and small molecules to obtain conjugates that self-assemble. (B) FESEM images of Ac-ILVAG-biotin reveal its nanofibrous architecture, confirming that functionalization at the C-terminus does not disrupt the nanofibrous architecture.

### DETAILED DESCRIPTION OF THE INVENTION

### Further Definitions

Unless defined otherwise, all technical and scientific terms used herein have the same meaning as commonly understood by one of ordinary skill in the art to which this invention belongs.

Although any methods and materials similar or equivalent to those described herein can be used in the practice or testing of the present invention, the preferred methods and materials are described.

The terms “peptoid” and “peptidomimetic” are used herein interchangeably and refer to molecules designed to mimic a peptide. Peptoids or peptidomimetics can arise either from modification of an existing peptide, or by designing similar systems that mimic peptides. These modifications involve changes to the peptide that will not occur naturally (such as altered backbones and/or the incorporation of non-natural amino acids). See above.

The term “amino acid” includes compounds in which the carboxylic acid group is shielded by a protecting group in the form of an ester (including an ortho ester), a silyl ester, an amide, a hydrazide, an oxazole, an 1,3-oxazoline or a 5-oxo-1,3,-oxazolidine. The term “amino acid” also includes compounds in which an amino group of the form —NH₂ or —NHR′ (supra) is shielded by a protecting group. Suitable amino protecting groups include, but are not limited to, a carbamate, an amide, a sulfonamide, an imine, an imide, histidine, a N-2,5,-dimethylpyrrole, an N-1,1,4,4-tetramethyldisilylazacyclopentane adduct, an N-1,1,3,3-tetramethyl-1,3-disilisoindoline, an N-diphenylsilyldiethylene, an 1,3,5-dioxazine, a N-[2-(trimethylsilyl)ethoxy]methylamine, a N-(5,5-dimethyl-3-oxo-1-cyclohexenyl)amine, a N-4,4,4-trifluoro-3-oxo-1-butenylamine, a N-9-borabicyclononane and a nitroamine. A protecting group may also be present that shields both the amino and the carboxylic group such as e.g. in the form of a 2,2-dimethyl-4-alkyl-2-sila-5-oxo-1,3-oxazolidine. The alpha carbon atom of the amino acid typically further carries a hydrogen atom. The so called “side chain” attached to the alpha carbon atom, which is in fact the continuing main chain of the carboxylic acid, is an aliphatic moiety that may be linear or branched. The term “side chain” refers to the presence of the amino acid in a peptide (supra), where a backbone is formed by coupling a plurality of amino acids. An aliphatic moiety bonded to the α carbon atom of an amino acid included in such a peptide then defines a side chain relative to the backbone. As explained above, the same applies to an aliphatic moiety bonded to the amino group of the amino acid, which likewise defines a side chain relative to the backbone of a peptoid.

The term “aliphatic” means, unless otherwise stated, a straight or branched hydrocarbon chain, which may be saturated or mono- or poly-unsaturated and include heteroatoms. The term “heteroatom” as used herein means an atom of any element other than carbon or hydrogen. An unsaturated aliphatic group contains one or more double and/or triple bonds (alkenyl or alkynyl moieties). The branches of the hydrocarbon chain may include linear chains as well as non-aromatic cyclic elements. The hydrocarbon chain, which may, unless otherwise stated, be of any length, and contain any number of branches. Typically, the hydrocarbon (main) chain includes 1 to 5, to 10, to 15 or to 20 carbon atoms. Examples of alkenyl radicals are straight-chain or branched hydrocarbon radicals which contain one or more double bonds. Alkenyl radicals generally contain about two to about twenty carbon atoms and one or more, for instance two, double bonds, such as about two to about ten carbon atoms, and one double bond. Alkynyl radicals normally contain about two to about twenty carbon atoms and one or more, for example two, triple bonds, preferably such as two to ten carbon atoms, and one triple bond. Examples of alkynyl radicals are straight-chain or branched hydrocarbon radicals which contain one or more triple bonds. Examples of alkyl groups are methyl, ethyl, propyl, butyl, pentyl, hexyl, heptyl, octyl, nonyl, decyl, the n isomers of these radicals, isopropyl, isobutyl, isopentyl, sec-butyl, tert-butyl, neopentyl, 3,3 dimethylbutyl. Both the main chain as well as the branches may furthermore contain heteroatoms as for instance N, O, S, Se or Si or carbon atoms may be replaced by these heteroatoms.

An aliphatic moiety may be substituted or unsubstituted with one or more functional groups. Substituents may be any functional group, as for example, but not limited to, amino, amido, azido, carbonyl, carboxyl, keto, cyano, isocyano, dithiane, halogen, hydroxyl, nitro, organometal, organoboron, seleno, silyl, silano, sulfonyl, thio, thiocyano, trifluoromethyl sulfonyl, p-toluenesulfonyl, bromobenzenesulfonyl, nitrobenzenesulfonyl, and methanesulfonyl.

As should be apparent from the above, the side chain of an amino acid in a peptide/peptoid described herein may be of a length of 0 to about 5, to about 10, to about 15 or to about 20 carbon atoms. It may be branched and include unsaturated carbon-carbon bonds. In some embodiments one or more natural amino acids are included in the peptide or peptoid. Such a natural amino acid may be one of the 20 building blocks of naturally occurring proteins.

In a peptide or peptoid, including a peptide/peptoid disclosed herein individual amino acids are covalently coupled via amide bonds between a carboxylic group of a first and an amino group of a second amino acid.

The term hydrophobic refers to a compound that is soluble in non-polar fluids. The hydrophobic properties of the peptide and/or peptoid are due to the presence of non-polar moieties within the same peptide and/or peptoid. Besides the hydrophobic peptide sequemce part there is a C-terminal —COOH moiety included that can occur in free, unprotected form, or in protected form. Non-polar moieties of a peptide or peptoid include a hydrocarbon chain that does not carry a functional group.

The non-polar moiety includes an amino acid, generally at least two amino acids, with a hydrocarbon chain that does not carry a functional group. The respective side chain, coupled to the α-carbon atom of the amino acid (supra), may have a main chain that includes 0 to about 20 or 1 to about 20, including 0 to about 15, 1 to about 15, 0 to about 10, 1 to about 10, 1 to about 5 or 0 to about 5 carbon atoms. The non-polar moiety may thus include an amino acid without side chain, i.e. glycine. The peptide and/or peptoid side chain may be branched (supra) and include one or more double or triple bonds (supra). Examples of peptide and/or peptoid side chains include, but are not limited to, methyl, ethyl, propyl, isopropyl, propenyl, propinyl, butyl, butenyl, sec-butyl, tert-butyl, isobutyl, pentyl, neopentyl, isopentyl, pentenyl, hexyl, 3,3 dimethylbutyl, heptyl, octyl, nonyl or decyl groups. As a few illustrative examples, the non-polar moiety may include an amino acid of alanine, valine, leucine, isoleucine, norleucine, norvaline, 2-(methylamino)-isobutyric acid, 2-amino-5-hexynoic acid. Such an amino acid may be present in any desired configuration. Bonded to the non-polar moiety may also be the C-terminus or the N-terminus of the peptide/peptoid. Typically the C-terminus or the N-terminus is in such a case shielded by a protecting group (supra).

In some embodiments the non-polar moiety includes a sequence of amino acids that is arranged in decreasing or increasing size. Hence, a portion of the amino acids of the non-polar moiety may be arranged in a general sequence of decreasing or increasing size. Relative to the direction from N- to C-terminus or from C- to N-terminus this general sequence can thus be taken to be of decreasing size. By the term “general sequence” of decreasing or increasing size is meant that embodiments are included in which adjacent amino acids are of about the same size as long as there is a general decrease or increase in size. Within a general sequence of decreasing size the size of adjacent amino acids of the non-polar moiety is accordingly identical or smaller in the direction of the general sequence of decreasing size. In some embodiments the general sequence of decreasing or increasing size is a non-repetitive sequence.

As an illustrative example, where a respective portion of amino acids is a sequence of five amino acids, the first amino acid may have a 3,4-dimethyl-hexyl side chain. The second amino acid may have a neopentyl side chain. The third amino acid may have a pentyl side chain. The fourth amino acid may have a butyl side chain. The fifth amino acid may be glycine, i.e. have no side chain. Although a neopentyl and a pentyl side chain are of the same size, the general sequence of such a non-polar peptide portion is decreasing in size. As a further illustrative example of a general sequence of decreasing size in a non-polar moiety the respective non-polar portion may be a sequence of three amino acids. The first amino acid may have an n-nonyl side chain. The second amino acid may have a 3-ethyl-2-methyl-pentyl side chain. The third amino acid may have a tert-butyl side chain. As yet a further illustrative example of a general sequence of decreasing size in a non-polar moiety, the non-polar moiety may be a sequence of nine amino acids. The first amino acid may have a 4-propyl-nonyl side chain. The second amino acid may have an n-dodecyl side chain. The third amino acid may have a 6,6-diethyl-3-octenyl side chain. An n-dodecyl side chain and a 6,6-diethyl-3-octenyl side chain both have 12 carbon atoms and thus again have a comparable size, Nevertheless, the 6,6-diethyl-3-octenyl group includes an unsaturated carbon-carbon bond and is thus of slightly smaller size than the dodecyl group. The fourth amino acid may have a 2-methyl-nonyl side chain. The fifth amino acid may have a 3-propyl-hexyl side chain. The sixth amino acid may have an n-hexyl side chain. The seventh amino acid may have a 2-butynyl side chain. The 8th amino acid may have an isopropyl side chain. The ninth amino acid may have a methyl side chain.

Where a portion of the amino acids of the non-polar moiety arranged in a general sequence of decreasing (or increasing) size only contains naturally occurring amino acids (whether in the D- or the L-form), it may for example have a length of five amino acids, such as the sequence leucine-isoleucine-valine-alanine-glycine or isoleucine-leucine-valine-alanine-glycine, A general sequence of decreasing size of only natural amino acids may also have a length of four amino acids. Illustrative examples include the sequences isoleucine-leucine-valine-alanine, leucine-isoleucine-valine-alanine, isoleucine-valine-alanine-glycine, leucine-valine-alanine-glycine, leucine-isoleucine-alanine-glycine, leucine-isoleucine-valine-glycine, isoleucine-leucine-alanine-glycine or isoleucine-leucine-valine-glycine. A general sequence of decreasing size of only natural amino acids may also have a length of three amino acids. Illustrative examples include the sequences isoleucine-valine-alanine, leucine-valine-alanine, isoleucine-valine-glycine, leucine-valine-glycine, leucine-alanine-glycine, isoleucine-alanine-glycine or isoleucine-leucine-alanine. A general sequence of decreasing size of only natural amino acids may also have a length of two amino acids. Illustrative examples include the sequences isoleucine-valine, leucine-valine, isoleucine-alanine, leucine-alanine, leucine-glycine, isoleucine-glycine, valine-alanine, valine-glycine or alanine-glycine.

In some embodiments the direction of decreasing size of the above defined general sequence of decreasing size is the direction toward the C-terminus of the hydrophobic linear sequence. Accordingly, in such embodiments the size of adjacent amino acids within this portion of the non-polar moiety is accordingly identical or smaller in the direction of the C-terminus. Hence, as a general trend in such an embodiment, the closer to the polar moiety of the amphiphilic linear sequence, the smaller is the overall size of a peptide and/or peptoid side chain throughout the respective general sequence of decreasing size.

In some embodiments the entire non-polar moiety of the hydrophobic linear peptide and/or peptoid or the hydrophobic linear sequence, respectively, consists of the general sequence of decreasing (or increasing) size. In such an embodiment the general sequence of decreasing (or increasing) size may have a length of n−m amino acids (cf. above). In some embodiments the general sequence of decreasing or increasing size is flanked by further non-polar side chains of the peptide/peptoid. In one embodiment the general sequence of decreasing (or increasing) size has a length of n−m−1 amino acids. In this embodiment there is one further amino acid included in the peptide/peptoid, providing a non-polar peptide/peptoid side chain. This amino acid may be positioned between the general sequence of decreasing (or increasing) size and the C-terminus, the C-terminus may be positioned between this additional non-polar amino acid and the general sequence of decreasing (or increasing) size or the general sequence of decreasing (or increasing) size may be positioned between the C-terminus and this additional non-polar amino acid. Typically the general sequence of decreasing (or increasing) size is positioned between the C-terminus and this additional non-polar amino acid. The additional non-polar amino acid may for example define the N-terminus of the peptide/peptoid, which may be shielded by a protecting group such as an amide, e.g. a propionic acyl or an acetyl group. Together with the general sequence of decreasing (or increasing) size as defined above it may define the non-polar portion of the peptide/peptoid. The polar amino acid may define the C-terminus of the peptide/peptoid. In this example the general sequence of decreasing (or increasing) size is thus flanked by the polar amino acid on one side and by the additional non-polar amino acid on the other side. In one embodiment where embodiment the general sequence of decreasing (or increasing) size has a length of n−m−1 amino acids, the remaining non-polar amino acid of the non-polar moiety of n−m amino acids is one of alanine and glycine.

As explained above, the polar moiety of the linear sequence may in some embodiments be defined by two or three consecutive amino acids. The polar moiety includes in aliphatic amino acids. Each of the in aliphatic amino acids is independently selected and carries an independently selected polar group. The symbol in represents an integer selected from 1, 2 and 3. The at least essentially non-polar moiety (supra) accordingly has a number of n−m, i.e. n−1, n−2 or n−3 amino acids. In some embodiments n is equal to or larger than m+2. In such an embodiment m may thus represent a number of n−2 or smaller.

In an embodiment where the entire non-polar moiety of the linear peptide and/or peptoid consists of the general sequence of decreasing (or increasing) size (supra), this non-polar moiety may thus have a length of n−2 or n−3 amino acids. In an embodiment where the linear peptide and/or peptoid has a further non-polar side chain in addition to the non-polar moiety of decreasing (or increasing) size, this additional non-polar side chain may be included in an amino acid that is directly bonded to an amino acid of the general sequence of decreasing (or increasing) size. The non-polar moiety may thus be defined by the non-polar moiety of decreasing (or increasing) size and the respective further amino acid with a non-polar side chain. In one such an embodiment where m=1, the non-polar moiety may thus have a length of n−2 amino acids, of which the non-polar moiety of decreasing (or increasing) size has a length of n−3 amino acids. The general sequence of decreasing (or increasing) size may be positioned between the two polar amino acids and this additional non-polar amino acid, or the additional non-polar amino acid may be positioned between the general sequence of decreasing (or increasing) size and the two polar amino acids. Typically the general sequence of decreasing (or increasing) size is positioned between the two polar amino acids and this additional non-polar amino acid. As mentioned above, one of the two polar amino acids may define the C-terminus of the peptide/peptoid. In this example the general sequence of decreasing (or increasing) size may thus be flanked by the two consecutive polar amino acids on one side and by the additional non-polar amino acid on the other side. Again, in some embodiments where m=1 the two consecutive polar amino acids may also be positioned between the general sequence of decreasing (or increasing) size and the additional non-polar amino acid, in which case the non-polar moiety has a first portion with a length of n−3 amino acids and a further portion of one amino acid.

Electrostatic forces, hydrogen bonding and van der Waals forces between hydrophobic linear sequences as defined above, including hydrophobic linear peptides and/or peptoids, result in these hydrophobic linear sequences to be coupled to each other. Without being bound by theory, thereby a cross-linking effect occurs that allows the formation of a hydrogel. In this regard the inventors have observed the formation of fibers based on helical structures.

The fibers formed of hydrophobic linear sequences of hydrophobic peptides and/or peptoids disclosed herein typically show high mechanical strength, which renders them particularly useful in tissue regeneration applications, for instance the replacement of damaged tissue. Hydrophobic peptides and/or peptoids disclosed herein have been observed to generally assemble into a fiber structure that resembles collagen fibers. Collagen, a component of soft tissue in the animal and human body, is a fibrous protein that provides most of the tensile strength of tissue. The mechanical strength of fibers of hydrophobic peptides and/or peptoids disclosed herein has been found to typically be much higher than that of collagen (cf. e.g. Figures) of gelatine, the hydrolysed form of collagen. An hydrophobic peptide and/or peptoid disclosed herein may thus be included in a hydrogel that is used as permanent or temporary prosthetic replacement for damaged or diseased tissue.

The hydrophobic linear sequence of the peptide/peptoid, which may represent the entire hydrophobic peptide/peptoid (supra) has been found to show remarkable stability at physiological conditions, even at elevated temperatures. It is in some embodiments stable in aqueous solution at physiological conditions at ambient temperature for a period of time in the range from 1 day to 1 month or more. It may in some embodiments be stable in aqueous solution at physiological conditions at 90° C. for at least 1 hour, at least 2 hours, at least 3 hours, at least 4 hours or at least 5 hours An hydrophobic linear sequence of an hydrophobic peptide and/or peptoid including an hydrophobic linear peptide and/or peptoid, is capable of providing a self assembling α-helical fiber in aqueous solution under physiological conditions. The peptides/peptoids (typically 3-7-mers) in the L- or D-form can self assemble into supramolecular helical fibers which are organized into mesh-like structures mimicking biological substances such as collagen. It has previously been observed in X-ray crystallography that peptides of a length of 3 to 6 amino acids with repetitive alanine containing sequences and an acetylated C-terminus take a helical conformation (Hatakeyama, Y, et al, Angew. Chem. Int. Ed. (2009) 8695-8698). Using peptides with an hydrophobic sequence, Ac-LD₆ (L), the formation of aggregates has for example been observed already at 0.1 mg/ml. As the concentration of peptide is increased to 1 mg/ml, the peptide monomers were found to align to form fibrous structures. With a formation of fibers occurring under physiological conditions at concentrations below 2 mM a peptide/peptoid is well suited as an injectable hydrogel material that can form a hydrogel under physiological conditions. Also disclosed herein is an hydrophobic linear peptide and/or peptoid as defined above for tissue engineering as well as to a tissue engineering method that involves applying, including injecting a respective hydrophobic linear peptide and/or peptoid.

A hydrogel is typically characterized by a remarkable rigidity and are generally biocompatible and non-toxic. Depending on the selected peptide/peptoid sequence these hydrogels can show thermoresponsive or thixotropic character. Reliant on the peptide/peptoid assembling conditions the fibers differ in thickness and length. Generally rigid hydrogels are obtained that are well suited for cultivation of a variety of primary human cells, providing peptide/peptoid scaffolds that can be useful in the repair and replacement of various tissues. Disclosed is also a process of preparing these hydrogels. The exemplary usage of these hydrogels in applications such as cell culture, tissue engineering, plastic surgery, drug delivery, oral applications, cosmetics, packaging and the like is described, as well as for technical applications, as for example for use in electronic devices which might include solar or fuel cells.

As an hydrophobic linear sequence of the peptide/peptoid, a hydrogel shows high stability at physiological conditions, even at elevated temperatures. In some embodiments such a hydrogel is stable in aqueous solution at ambient temperature for a period of at least 7 days, at least 14 days, at least a month or more, such as at least 1 to about 6 months.

In some embodiments a hydrogel disclosed herein is coupled to a molecule or a particle, including a quantum dot, with characteristic spectral or fluorometric properties, such as a marker, including a fluorescent dye. A respective molecule may for instance allow monitoring the fate, position and/or the integrity of the hydrogel.

In some embodiments a hydrogel disclosed herein is coupled to a molecule with binding affinity for a selected target molecule, such as a microorganism, a virus particle, a peptide, a peptoid, a protein, a nucleic acid, a peptide, an oligosaccharide, a polysaccharide, an inorganic molecule, a synthetic polymer, a small organic molecule or a drug.

The term “nucleic acid molecule” as used herein refers to any nucleic acid in any possible configuration, such as single stranded, double stranded or a combination thereof. Nucleic acids include for instance DNA molecules (e.g., cDNA or genomic DNA), RNA molecules (e.g., mRNA), analogues of the DNA or RNA generated using nucleotide analogues or using nucleic acid chemistry, locked nucleic acid molecules (LNA), and protein nucleic acids molecules (PNA). DNA or RNA may be of genomic or synthetic origin and may be single or double stranded. In the present method of an embodiment of the invention typically, but not necessarily, an RNA or a DNA molecule will be used. Such nucleic acid can be e.g. mRNA, cRNA, synthetic RNA, genomic DNA, cDNA synthetic DNA, a copolymer of DNA and RNA, oligonucleotides, etc. A respective nucleic acid may furthermore contain non-natural nucleotide analogues and/or be linked to an affinity tag or a label. In some embodiments the nucleic acid molecule may be isolated, enriched, or purified. The nucleic acid molecule may for instance be isolated from a natural source by cDNA cloning or by subtractive hybridization. The natural source may be mammalian, such as human, blood, semen, or tissue. The nucleic acid may also be synthesized, e.g. by the triester method or by using an automated DNA synthesizer.

Many nucleotide analogues are known and can be used in nucleic acids and oligonucleotides used in the methods of exemplary embodiments of the invention. A nucleotide analogue is a nucleotide containing a modification at for instance the base, sugar, or phosphate moieties. Modifications at the base moiety include natural and synthetic modifications of A, C, G, and T/U, different purine or pyrimidine bases, such as uracil-5-yl, hypoxanthin-9-yl, and 2-aminoadenin-9-yl, as well as non-purine or non-pyrimidine nucleotide bases. Other nucleotide analogues serve as universal bases. Universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

A peptide may be of synthetic origin or isolated from a natural source by methods well-known in the art. The natural source may be mammalian, such as human, blood, semen, or tissue. A peptide, including a polypeptide may for instance be synthesized using an automated polypeptide synthesizer. Illustrative examples of polypeptides are an antibody, a fragment thereof and a proteinaceous binding molecule with antibody-like functions. Examples of (recombinant) antibody fragments are Fab fragments, Fv fragments, single-chain Fv fragments (scFv), diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). An example of a proteinaceous binding molecule with antibody-like functions is a mutein based on a polypeptide of the lipocalin family (WO 03/029462, Beste et al., Proc. Natl. Acad. Sci. U.S.A. (1999) 96, 1898-1903). Lipocalins, such as the bilin binding protein, the human neutrophil gelatinase-associated lipocalin, human Apolipoprotein D or glycodelin, posses natural ligand-binding sites that can be modified so that they bind to selected small protein regions known as haptens. Examples of other proteinaceous binding molecules are the so-called glubodies (see e.g. internation patent application WO 96/23879), proteins based on the ankyrin scaffold (Mosavi, L. K., et al., Protein Science (2004) 13, 6, 1435-1448) or crystalline scaffold (e.g. internation patent application WO 01/04144) the proteins described in Skerra, J. Mol. Recognit. (2000) 13, 167-187, AdNectins, tetranectins and avimers. Avimers contain so called A-domains that occur as strings of multiple domains in several cell surface receptors (Silverman, J., et al., Nature Biotechnology (2005) 23, 1556-1561). Adnectins, derived from a domain of human fibronectin, contain three loops that can be engineered for immunoglobulin-like binding to targets (Gill, D. S. & Damle, N. K., Current Opinion in Biotechnology (2006) 17, 653-658). Tetranectins, derived from the respective human homotrimeric protein, likewise contain loop regions in a C-type lectin domain that can be engineered for desired binding (ibid.). Where desired, a modifying agent may be used that further increases the affinity of the respective moiety for any or a certain form, class etc. of target matter.

An example of a nucleic acid molecule with antibody-like functions is an aptamer. An aptamer folds into a defined three-dimensional motif and shows high affinity for a given target structure. Using standard techniques of the art such as solid-phase synthesis an aptamer with affinity to a certain target can accordingly be formed and immobilized on a hollow particle of an embodiment of the invention.

As a further illustrative example, a linking moiety such as an affinity tag may be used to immobilise the respective molecule. Such a linking moiety may be a molecule, e.g. a hydrocarbon-based (including polymeric) molecule that includes nitrogen-, phosphorus-, sulphur-, carben-, halogen- or pseudohalogen groups, or a portion thereof. As an illustrative example, the peptide/peptoid included in the hydrogel may include functional groups, for instance on a side chain of the peptide/peptoid, that allow for the covalent attachment of a biomolecule, for example a molecule such as a protein, a nucleic acid molecule, a polysaccharide or any combination thereof. A respective functional group may be provided in shielded form, protected by a protecting group that can be released under desired conditions. Examples of a respective functional group include, but are not limited to, an amino group, an aldehyde group, a thiol group, a carboxy group, an ester, an anhydride, a sulphonate, a sulphonate ester, an imido ester, a silyl halide, an epoxide, an aziridine, a phosphoramidite and a diazoalkane.

Examples of an affinity tag include, but are not limited to, biotin, dinitrophenol or digoxigenin, oligohistidine, polyhistidine, an immunoglobulin domain, maltose-binding protein, glutathione-S-transferase (GST), calmodulin binding peptide (CBP), FLAG′-peptide, the T7 epitope (Ala-Ser-Met-Thr-Gly-Gly-Gln-Gln-Met-Gly), maltose binding protein (MBP), the HSV epitope of the sequence Gln-Pro-Glu-Leu-Ala-Pro-Glu-Asp-Pro-Glu-Asp of herpes simplex virus glycoprotein D, the hemagglutinin (HA) epitope of the sequence Tyr-Pro-Tyr-Asp-Val-Pro-Asp-Tyr-Ala, the “myc” epitope of the transcription factor c-myc of the sequence Glu-Gln-Lys-Leu-Ile-Ser-Glu-Glu-Asp-Leu, or an oligonucleotide tag. Such an oligonucleotide tag may for instance be used to hybridise to an immobilised oligonucleotide with a complementary sequence. A further example of a linking moiety is an antibody, a fragment thereof or a proteinaceous binding molecule with antibody-like functions (see also above).

A further example of linking moiety is a cucurbituril or a moiety capable of forming a complex with a cucurbituril. A cucurbituril is a macrocyclic compound that includes glycoluril units, typically self-assembled from an acid catalyzed condensation reaction of glycoluril and formaldehyde. A cucurbit[n]uril, (CB[n]), that includes n glycoluril units, typically has two portals with polar ureido carbonyl groups. Via these ureido carbonyl groups cucurbiturils can bind ions and molecules of interest. As an illustrative example cucurbit[7]uril (CB[7]) can form a strong complex with ferrocenemethylammonium or adatnantylarnmonium ions. Either the cucurbit[7]uril or e.g. ferrocenemethylammonium may be attached to a biomolecule, while the remaining binding partner (e.g. ferrocenemethylammonium or cucurbit[7]uril respectively) can be bound to a selected surface. Contacting the biomolecule with the surface will then lead to an immobilisation of the biomolecule. Functionalised CB[7] units bound to a gold surface via alkanethiolates have for instance been shown to cause an immobilisation of a protein carrying a ferrocenemethylammonium unit (Hwang, I., et al., J. Am. Chem. Soc. (2007) 129, 4170-4171).

Further examples of a linking moiety include, but are not limited to an oligosaccharide, an oligopeptide, biotin, dinitrophenol, digoxigenin and a metal chelator (cf. also below). As an illustrative example, a respective metal chelator, such as ethylenediamine, ethylenediamine-tetraacetic acid (EDTA), ethylene glycol tetraacetic acid (EGTA), diethylenetriaminepentaacetic acid (DTPA), N,N-bis(carboxymethyl)glycine (also called nitrilotriacetic acid, NTA), 1,2-bis(o-aminophenoxy)ethane-N,N,N′,N′-tetraacetic acid (BAPTA), 2,3-dimercapto-1-propanol (dimercaprol), porphine or heme may be used in cases where the target molecule is a metal ion. As an example, EDTA forms a complex with most monovalent, divalent, trivalent and tetravalent metal ions, such as e.g. silver (Ag⁺), calcium (Ca²⁺), manganese (Mn²⁺), copper (Cu²⁺), iron (Fe²⁺), cobalt (Co³⁺) and zirconium (Zr⁴⁺), while BAPTA is specific for Ca²⁺. In some embodiments a respective metal chelator in a complex with a respective metal ion or metal ions defines the linking moiety. Such a complex is for example a receptor molecule for a peptide of a defined sequence, which may also be included in a protein. As an illustrative example, a standard method used in the art is the formation of a complex between an oligohistidine tag and copper (Cu²⁺), nickel (Ni²⁺), cobalt (Co²⁺), or zink (Zn²⁺) ions, which are presented by means of the chelator nitrilotriacetic acid (NTA).

Avidin or streptavidin may for instance be employed to immobilise a biotinylated nucleic acid, or a biotin containing monolayer of gold may be employed (Shumaker-Parry, J. S., et al., Anal. Chem. (2004) 76, 918). As yet another illustrative example, the biomolecule may be locally deposited, e.g. by scanning electrochemical microscopy, for instance via pyrrole-oligonucleotide patterns (e.g. Fortin, E., et al., Electroanalysis (2005) 17, 495). In other embodiments, in particular where the biomolecule is a nucleic acid, the biomolecule may be directly synthesised on the surface of the immobilisation unit, for example using photoactivation and deactivation. As an illustrative example, the synthesis of nucleic acids or oligonucleotides on selected surface areas (so called “solid phase” synthesis) may be carried out using electrochemical reactions using electrodes. An electrochemical deblocking step as described by Egeland & Southern (Nucleic Acids Research (2005) 33, 14, e125) may for instance be employed for this purpose. A suitable electrochemical synthesis has also been disclosed in US patent application US 2006/0275927. In some embodiments light-directed synthesis of a biomolecule, in particular of a nucleic acid molecule, including UV-linking or light dependent 5′-deprotection, may be carried out.

The molecule that has a binding affinity for a selected target molecule may be immobilised on the nanocrystals by any means. As an illustrative example, an oligo- or polypeptide, including a respective moiety, may be covalently linked to the surface of nanocrystals via a thio-ether-bond, for example by using ω functionalized thiols. Any suitable molecule that is capable of linking a nanocrystal of an embodiment of the invention to a molecule having a selected binding affinity may be used to immobilise the same on a nanocrystal. For instance a (bifunctional) linking agent such as ethyl-3-dimethylaminocarbodiimide, N-(3-aminopropyl) 3-mercapto-benzamide, 3-aminopropyl-trimethoxysilane, 3-mercaptopropyl-trimethoxysilane, 3-(trimethoxysilyl) propyl-maleimide, or 3-(trimethoxysilyl) propyl-hydrazide may be used. Prior to reaction with the linking agent, the surface of the nanocrystals can be modified, for example by treatment with glacial mercaptoacetic acid, in order to generate free mercaptoacetic groups which can then employed for covalently coupling with an analyte binding partner via linking agents.

Embodiments of the present invention also include a hydrogel, which can be taken to be a water-swollen water-insoluble polymeric material. The hydrogel includes, including contains and consists of a peptide and/or peptoid as defined above. Since a hydrogel maintains a three-dimensional structure, a hydrogel of an embodiment of the invention may be used for a variety of applications. Since the hydrogel has a high water content and includes amino acids, it is typically of excellent biocompatibility.

A hydrogel according to an embodiment of the invention is formed by self-assembly. The inventors have observed that the peptides/peptoids assemble into fibers that form mesh-like structures. Without being bound by theory hydrophobic interaction between non-polar portions of peptides/peptoids are contemplated to assist such self-assembly process.

The method of forming the hydrogel includes dissolving the peptide/peptoid in aqueous solution. Agitation, including mixing such as stirring, and/or sonication may be employed to facilitate dissolving the peptide/peptoid. In some embodiments the aqueous solution with the peptide/peptoid therein is exposed to a temperature below ambient temperature, such as a temperature selected from about 2° C. to about 15° C. In some embodiments the aqueous solution with the peptide/peptoid therein is exposed to an elevated temperature, i.e. a temperature above ambient temperature. Typically the aqueous solution is allowed to attain the temperature to which it is exposed. The aqueous solution may for example be exposed to a temperature from about 25° C. to about 85° C. or higher, such as from about 25° C. to about 75° C., from about 25° C. to about 70° C., from about 30° C. to about 70° C., from about 35° C. to about 70° C., from about 25° C. to about 60° C., from about 30° C. to about 60° C., from about 25° C. to about 50° C., from about 30° C. to about 50° C. or from about 40° C. to about 65° C., such as e.g. a temperature of about 40° C., about 45° C., about 50° C., about 55° C., about 60° C. or about 65° C. The aqueous solution with the peptide/peptoid therein may be maintained at this temperature for a period of about 5 min to about 10 hours or more, such as about 10 min to about 6 hours, about 10 min to about 4 hours, about 10 min to about 2.5 hours, about 5 min to about 2.5 hours, about 10 min to about 1.5 hours or about 10 min to about 1 hour, such as about 15 min, about 20 min, about 25 min, about 30 min, about 35 min or about 40 min.

In some embodiments a hydrogel disclosed herein is a biocompatible, including a pharmaceutically acceptable hydrogel. The term “biocompatible” (which also can be referred to as “tissue compatible”), as used herein, is a hydrogel that produces little if any adverse biological response when used in vivo. The term thus generally refers to the inability of a hydrogel to promote a measurably adverse biological response in a cell, including in the body of an animal, including a human. A biocompatible hydrogel can have one or more of the following properties: non-toxic, non-mutagenic, non-allergenic, non-carcinogenic, and/or non-irritating. A biocompatible hydrogel, in the least, can be innocuous and tolerated by the respective cell and/or body. A biocompatible hydrogel, by itself, may also improve one or more functions in the body.

Depending on the amino acids that are included in the peptide/peptoid that is included in a hydrogel, a respective hydrogel may be biodegradable. A biodegradable hydrogel gradually disintegrates or is absorbed in vivo over a period of time, e.g., within months or years. Disintegration may for instance occur via hydrolysis, may be catalysed by an enzyme and may be assisted by conditions to which the hydrogel is exposed in a human or animal body, including a tissue, a blood vessel or a cell thereof. Where a peptide is made up entirely of natural amino acids, a respective peptide can usually be degraded by enzymes of the human/animal body.

A hydrogel according to an embodiment of the invention may also serve as a depot for a pharmaceutically active compound such as a drug. A hydrogel according to an embodiment of the invention may be designed to mimic the natural extracellular matrix of an organism such as the human or animal body. A fiber formed from the peptide/peptoid of an embodiment of the invention, including a respective hydrogel, may serve as a biological scaffold. A hydrogel of an embodiment of the invention may be included in an implant, in a contact lens or may be used in tissue engineering. In one embodiment, the peptides consist typically of 3-7 amino acids and are able to self-assemble into complex fibrous scaffolds which are seen as hydrogels, when dissolved in water or aqueous solution. These hydrogels can retain water up to 99.9% and possess sufficiently high mechanical strength. Thus, these hydrogels can act as artificial substitutes for a variety of natural tissues without the risk of immunogenicity. The hydrogels in accordance with the present invention may be used for cultivating suitable primary cells and thus establish an injectable cell-matrix compound in order to implant or reimplant the newly formed cell-matrix in vivo. Therefore, the hydrogels in accordance with the present invention are particularly useful for tissue regeneration or tissue engineering applications. As used herein, a reference to an “implant” or “implantation” refers to uses and applications of/for surgical or arthroscopic implantation of a hydrogel containing device into a human or animal, e.g. mammalian, body or limb. Arthroscopic techniques are taken herein as a subset of surgical techniques, and any reference to surgery, surgical, etc., includes arthroscopic techniques, methods and devices. A surgical implant that includes a hydrogel according to an embodiment of the invention may include a peptide and/or peptoid scaffold. This the peptide and/or peptoid scaffold may be defined by the respective hydrogel. A hydrogel of an embodiment of the invention may also be included in a wound cover such as gauze or a sheet, serving in maintaining the wound in a moist state to promote healing.

Depending on the amino acid sequence used in the peptide/peptoid the hydrogel may be temperature-sensitive. It may for instance have a lower critical solution temperature or a temperature range corresponding to such lower critical solution temperature, beyond which the gel collapses as hydrogen bonds by water molecules are released as water molecules are released from the gel.

The disclosed subject matter also provides improved chiral hydrophobic natural-based peptides and/or peptoids that assemble to peptide/peptoid hydrogels with very favorable material properties. The advantage of these peptide/peptoid hydrogels is that they are accepted by a variety of different primary human cells, thus providing peptide scaffolds that can be useful in the repair and replacement of various tissues. Depending on the chirality of the peptide monomer the character of the hydrogels can be designed to be more stable and less prone to degradation though still biocompatible.

A hydrogel and/or a peptide/peptoid described herein can be administered to an organism, including a human patient per se, or in pharmaceutical compositions where it may include or be mixed with pharmaceutically active ingredients or suitable carriers or excipient(s). Techniques for formulation and administration of respective hydrogels or peptides/peptoids resemble or are identical to those of low molecular weight compounds well established in the art. Exemplary routes include, but are not limited to, oral, transdermal, and parenteral delivery. A hydrogel or a peptide/peptoid may be used to fill a capsule or tube, or may be provided in compressed form as a pellet. The peptide/peptoid or the hydrogel may also be used in injectable or sprayable form, for instance as a suspension of a respective peptide/peptoid.

A hydrogel of an embodiment of the invention may for instance be applied onto the skin or onto a wound. Further suitable routes of administration may, for example, include depot, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections. It is noted in this regard that for administering microparticles a surgical procedure is not required. Where the microparticles include a biodegradable polymer there is no need for device removal after release of the anti-cancer agent. Nevertheless the microparticles may be included in or on a scaffold, a coating, a patch, composite material, a gel or a plaster.

In some embodiments one may administer a hydrogel and/or a peptide/peptoid in a local rather than systemic manner, for example, via injection.

Pharmaceutical compositions that include a hydrogel and/or a peptide/peptoid of an embodiment of the present invention may be manufactured in a manner that is itself known, e. g., by means of conventional mixing, dissolving, granulating, dragee-making, levigating, emulsifying, encapsulating, entrapping or lyophilizing processes.

Pharmaceutical compositions for use in accordance with an embodiment of the present invention thus may be formulated in conventional manner using one or more physiologically acceptable carriers including excipients and auxiliaries that facilitate processing of the hydrogel and/or peptide/peptoid into preparations that can be used pharmaceutically. Proper formulation is dependent upon the route of administration chosen.

For injection, the peptide/peptoid of an embodiment of the invention may be formulated in aqueous solutions, for instance in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the hydrogel and/or peptide/peptoid can be formulated readily by combining them with pharmaceutically acceptable carriers well known in the art. Such carriers enable the hydrogel and/or peptide/peptoid, as well as a pharmaceutically active compound, to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatine, as well as soft, sealed capsules made of gelatine and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the peptides/peptoids may be suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

The hydrogel and/or peptide/peptoid may be formulated for parenteral administration by injection, e.g., by intramuscular injections or bolus injection or continuous infusion. Formulations for injection may be presented in unit dosage form, e. g., in ampules or in multi-dose containers, with an added preservative. The respective compositions may take such forms as suspensions, solutions or emulsions in oily or aqueous vehicles, and may contain formulatory agents such as suspending, stabilizing and/or dispersing agents.

The hydrogel and/or peptide/peptoid may be formulated for other drug delivery systems like implants, or trandermal patches or stents.

The present invention provides a novel class of hydrogel-forming hydrophobic peptides/peptidomimetics.

The inventors have found advantages and properties that the absence of a polar head group, such as hydrophilic amino acid(s), is giving to small peptides consisting solely of hydrophobic amino acids.

The absence of a polar group at the C-terminus gives rise to a new class of self-assembling peptides with different properties to the so far disclosed class of ultrashort peptides. It is not evident for a person aware of the state-of-the-art that a solely hydrophobic sequence of amino acids will be able to self-assemble to fibrous scaffolds, ending up in hydrogels. The so far explored assembly process of the currently explored type of ultrashort peptides was thought to be solely depending on amphiphilic sequences. The absence of a polar head group would have been more likely predicted to generate micelle-like structures, but not soft solid material. In addition, the absence of a polar head group leads to new material properties and gives so far unexplored possibilities to create novel smart biomaterial.

New advantages in material properties can be designed by the functionalization via the conjugation of non-amino acids such as small molecules, functional groups and short linkers.

These small molecule/functional group/short linkers bestow new material properties such as bio-adhesiveness and receptor-targeting. The new peptide sequence characteristics enables the development of new (and different to the one developed so far) applications. It also simplifies the purification of the desired compound. Compared to the peptide itself, the presence of the functional group/short linker at the C-terminus enhances ease of functionalization and the ability to chemically conjugate multiple bioactive molecules (such as cytokines, prodrugs etc) to a single peptidomimetic/peptidic conjugate. We can also eliminate undesired side reactions and non-specific interactions between the peptidomimetic/peptidic conjugate and bioactive molecules of interest.

In a further aspect, the present invention provides the use of said hydrophobic peptides/peptidomimetics in biofabrication.

Peptide self-assembly is an elegant and expedient “bottom-up” approach towards designing ordered, three-dimensional nanobiomaterials. Reproducible macromolecular nanostructures can be obtained due to the highly specific interactions that govern self-assembly. The amino acid sequence determines peptide secondary structure and interactions with other molecules, which in turn dictates the higher order macromolecular architecture.

Self-assembled nanofibrillar peptide scaffolds are of great interest for applications in regenerative medicine. As their nanofibrous topography resembles the extracellular matrix, they have been extensively applied as biomimetic scaffolds, providing spatial and temporal cues to regulate cell growth and behavior. Spatially defined, large-scale three-dimensional scaffolds, incorporating cells and other biochemical cues, can be obtained by 3D microdroplet bio-printing and moulding techniques. Self-assembling peptides, peptidomimetics and peptidic conjugates can serve as building blocks for printing or moulding of biocompatible macromolecular scaffolds that support the growth of encapsulated cells.

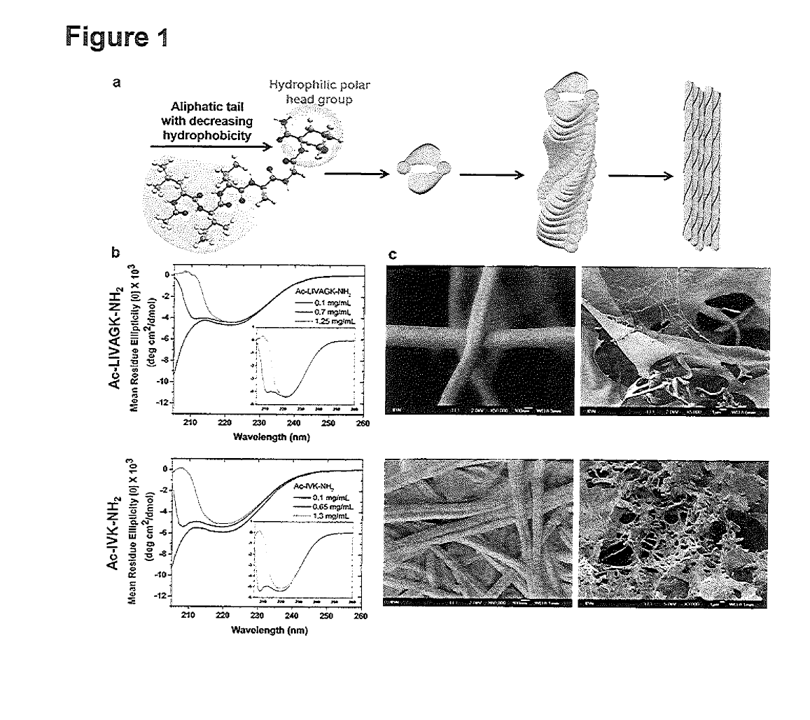

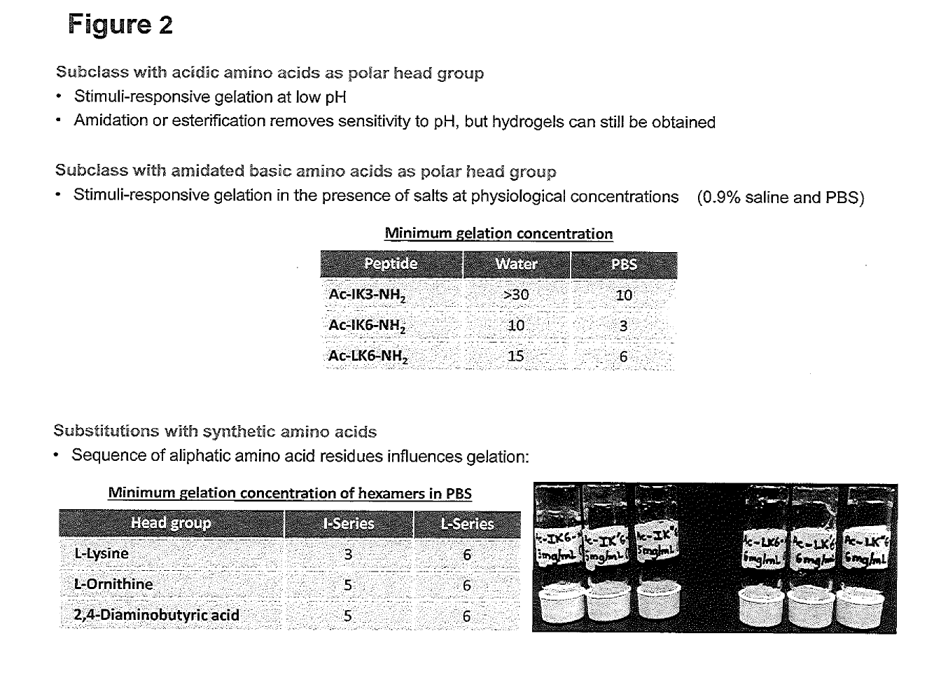

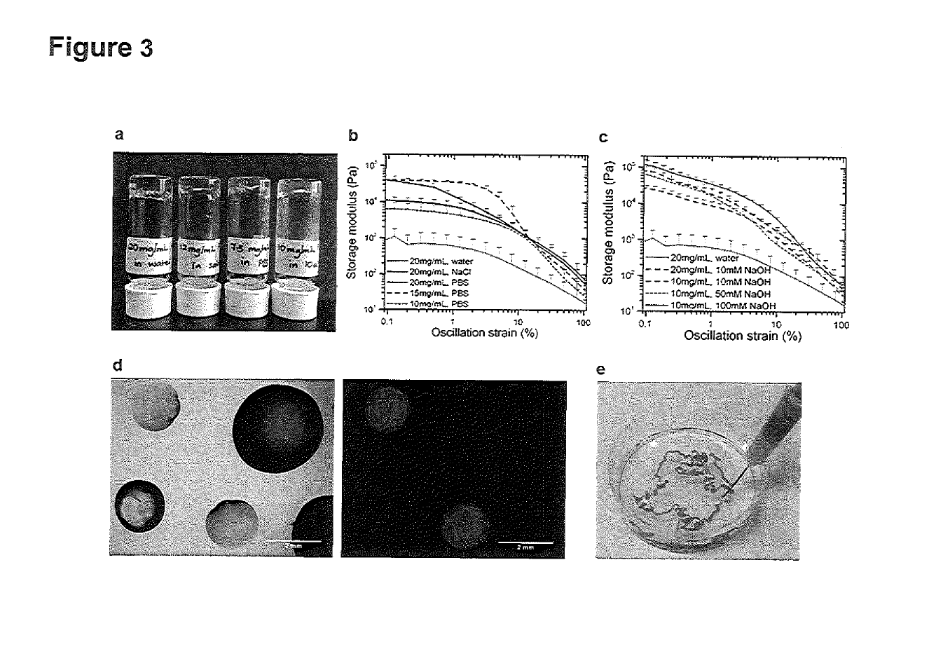

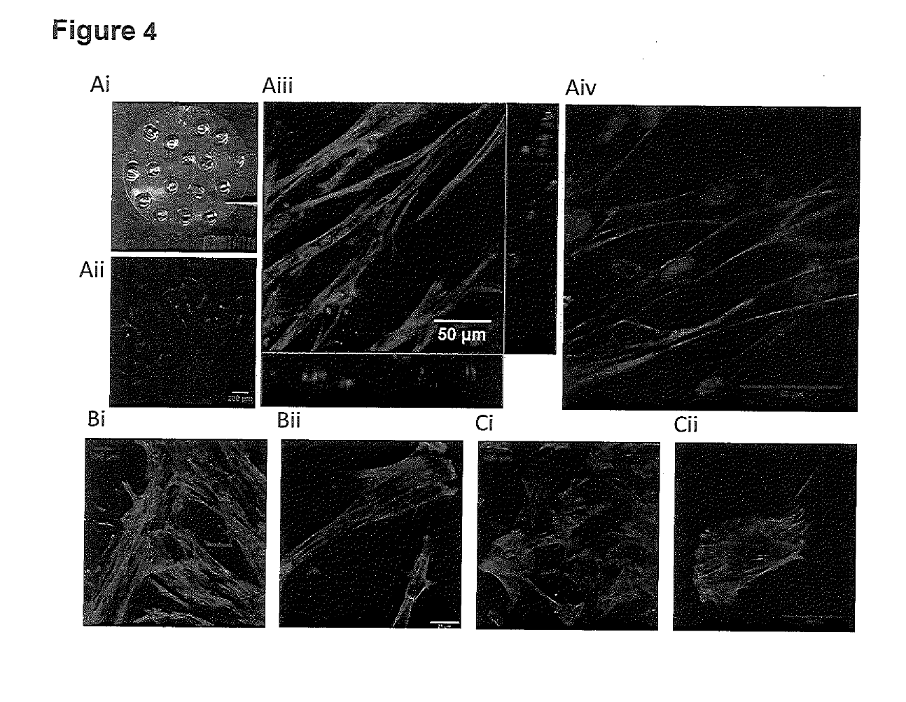

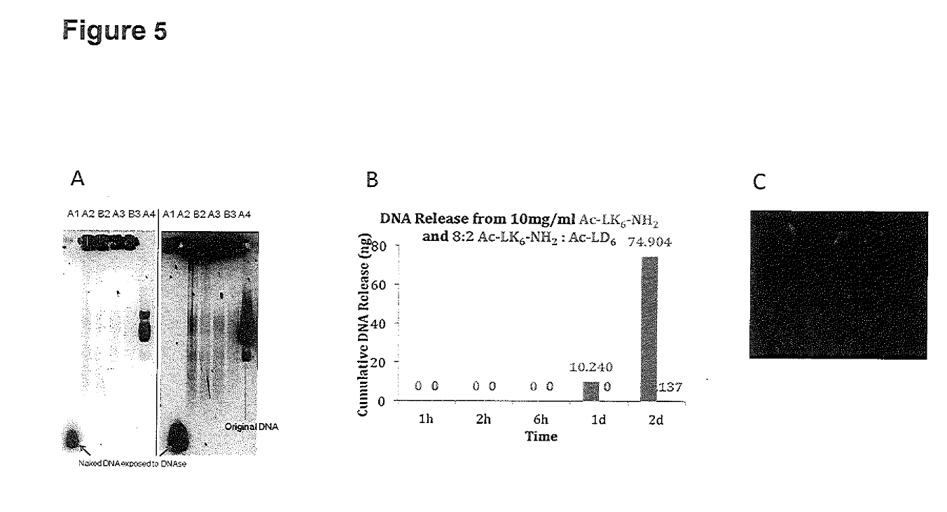

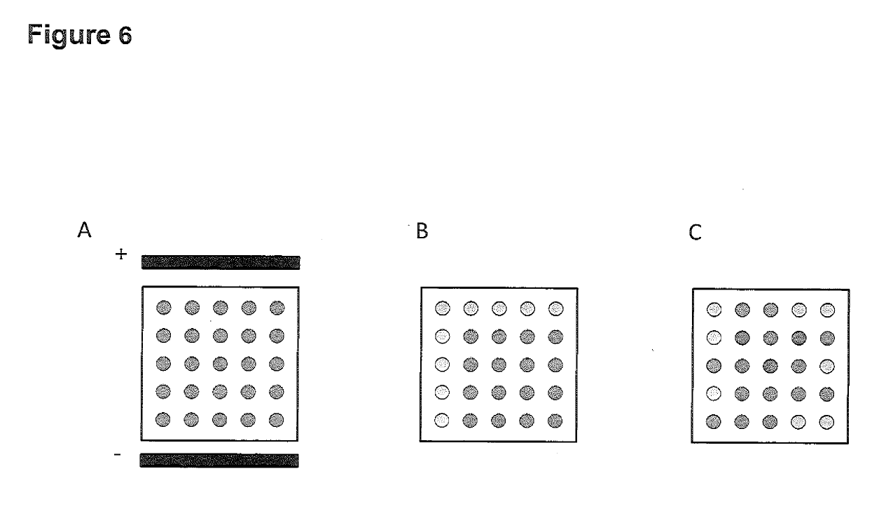

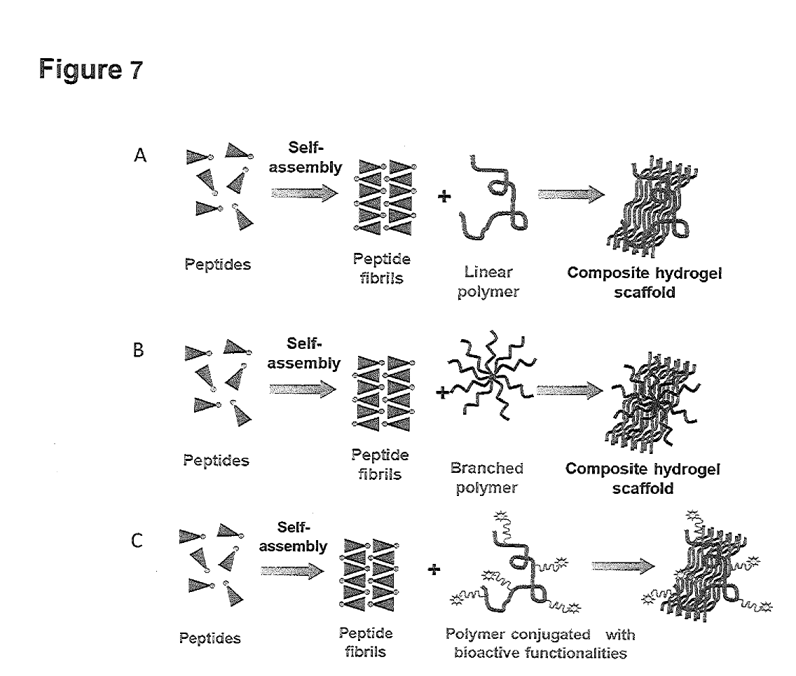

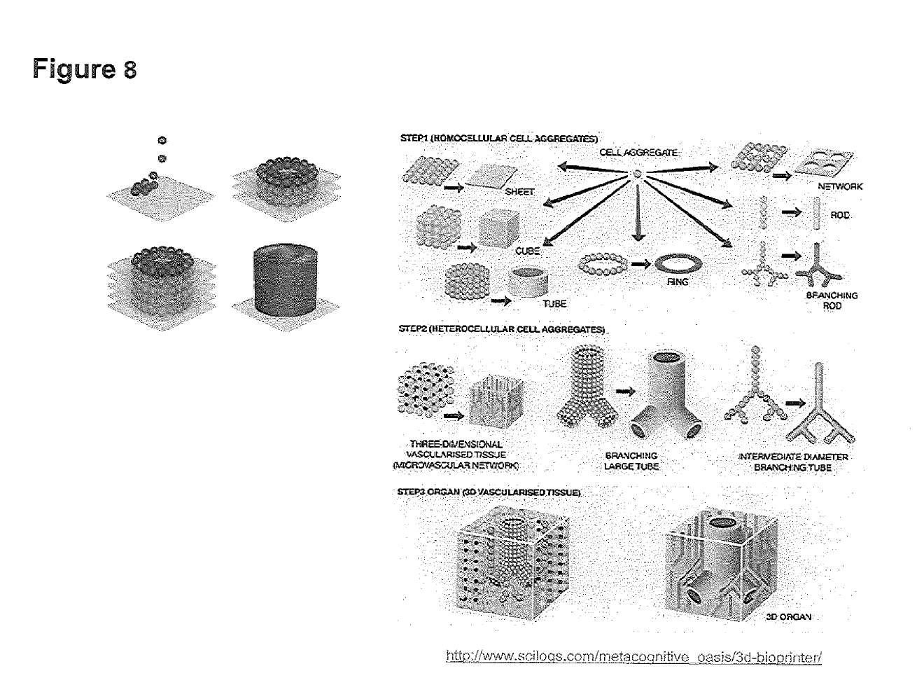

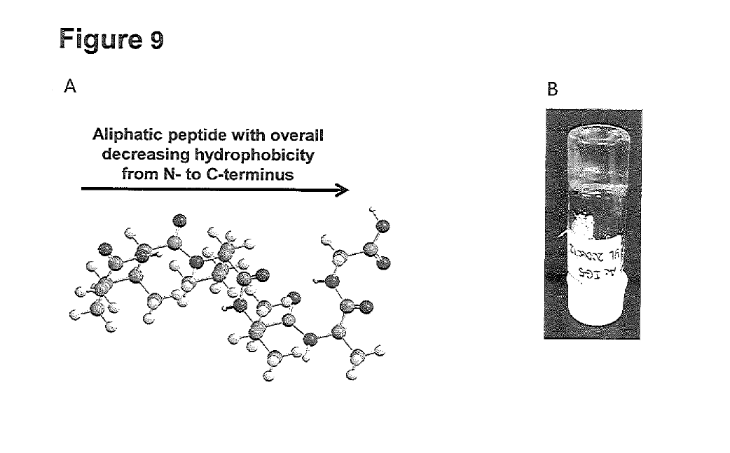

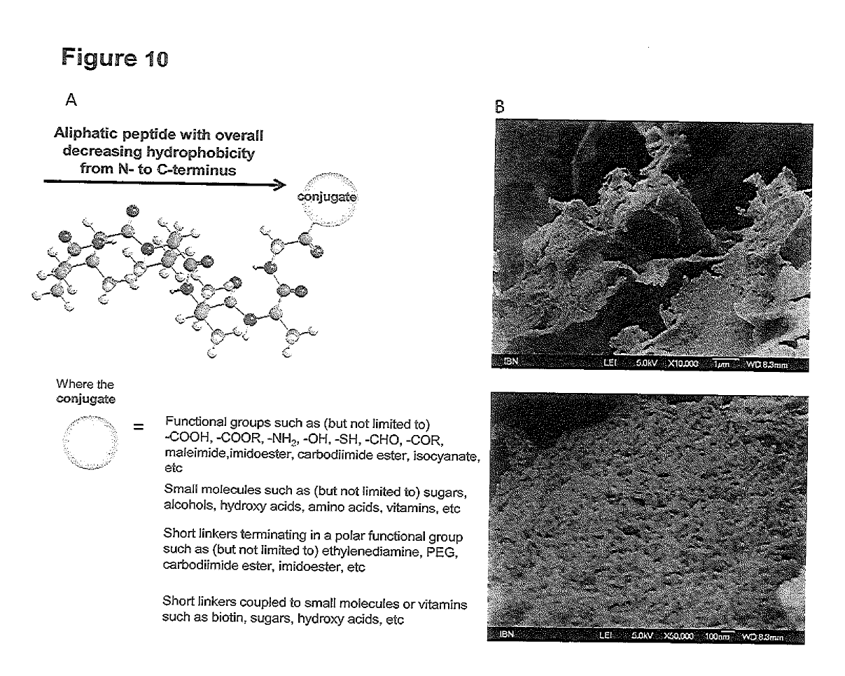

This disclosure describes a novel class of ultrashort peptides/peptidomimetics/conjugates, with a characteristic motif that facilitates self-assembly in aqueous conditions, forming porous, nanofibrous scaffolds that are biocompatible (FIG. 1). Several subclasses demonstrate stimuli-responsive gelation (FIG. 2) and can be used to for bio-printing of mini-hydrogel arrays and 3D organotypic biological constructs. The stimuli-responsive nature can also be exploited to produce hydrogel fibers or “noodles” through extrusion into salt solution baths. The resulting fibers can potentially be collected and used to create woven and aligned fibrous scaffolds.

The characteristic motif that drives self-assembly consists of a N-terminus “tail” of 2 to 7 natural aliphatic amino acids, arranged in decreasing hydrophobicity towards the C-terminus (FIG. 10). The C-terminus can be functionalized, such as with a functional group (e.g. carboxylic acid, amine, ester, alcohol, aldehyde, ketone, maleimide), small molecules (e.g. sugars, alcohols, vitamins, hydroxyl-acids, amino acids) or short polar linkers.

Self-assembly in aqueous conditions occurs when the amino acids pair and subsequently stack into α-helical fibrils (FIG. 1). Hydrogels are obtained when further aggregation of the fibrils into 3D networks of nanofibers entrap water (FIG. 3A).

The presence of functional groups enables to perform chemical modifications pre- and post-assembly. For instance, bioactive moieties such as growth factors, lipids, cell-receptor ligands, hormones and drugs can be conjugated to the scaffold post-assembly, giving rise to functionalized hydrogels.

Several subclasses of these peptides/peptidomimetics/conjugates demonstrate stimuli-responsive gelation (FIG. 2). In particular, a subclass of peptides with lysine or lysine-mimetic molecules as the polar head group exhibit enhanced gelation and rigidity in the presence of salts and elevated pH (FIGS. 3A, B and C). The gelation duration can be tuned by titrating the peptide and salt concentration. This opens avenues for the development of bio-printing, wherein gelation can be controlled and limited to desired areas through the co-injection of salt solutions.

Furthermore, the gelation process is slightly endodermic, which adds an element of temperature-sensitivity and eliminates the possibility of thermal damage to encapsulated cells. During the process of gelation, the ability to modulate gelation duration enables to sculpt the hydrogel construct into the desired shape for applications in regenerative medicine. The mechanical properties of this subclass of peptide hydrogels are enhanced by increasing salt concentration and pH. The stiffness and tunable mechanical properties render this subclass of amidated peptides hydrogels as ideal candidates for developing biological constructs that fulfill mechanically supportive roles. Through the judicious addition of ionic buffers and bases, less peptide can be used to attain equivalent mechanical stiffness while maintaining the porosity for supporting cell migration. The ability to modulate the mechanical properties and porosity is integral to creating organotypic constructs with mechanical properties comparable to that of the native tissue. In comparison, other peptide hydrogels, based on self-assembling α-helices, β-hairpins (G′≦2 kPa) and β-sheets (G′≦2 kPa), cannot attain such high rigidity. (References: α-helices:
Banwell, E. F. et al. Rational design and application of responsive alpha-helical peptide hydrogels. Nat Mater 8, 596-600 (2009).
Yan, C. & Pochan, D. J. Rheological properties of peptide-based hydrogels for biomedical and other applications. Chem Soc Rev 39, 3528-3540 (2010).

### β-Hairpins:

Yan, C. et al. Injectable solid hydrogel: mechanism of shear-thinning and immediate recovery of injectable β-hairpin peptide hydrogels. Soft Matter 6, 5143 (2010).
Schneider, J. P. et al. Responsive hydrogels from the intramolecular folding and self-assembly of a designed peptide. J Am Chem Soc 124, 15030-15037 (2002).

### References: β-Sheets:

Zhang, S., Holmes, T., Lockshin, C. & Rich, A. Spontaneous assembly of a self-complementary oligopeptide to form a stable macroscopic membrane. Proc. Natl. Acad. Sci. USA 90, 3334-3338 (1993).
Liu, J., Zhang, L., Yang, Z. & Zhao, X. Controlled release of paclitaxel from a self-assembling peptide hydrogel formed in situ and antitumor study in vitro. Int J Nanomedicine 6, 2143-2153 (2011).
Aggeli, A. et al. Responsive gels formed by the spontaneous self-assembly of peptides into polymeric beta-sheet tapes. Nature 386, 259-262 (1997).)

As a proof-of-concept, this subclass of peptides was used to demonstrate the feasibility of bio-printing to develop mini-hydrogel arrays and 3D organoid structures for screening and regenerative medicine. This subclass of peptides demonstrates good solubility in water, forming solutions with low viscosity. This facilitates the printing and prevents the clogging of the needle/printer. Upon interacting with a physiological salt solution (such as phosphate buffered saline, PBS), the peptide solution gels instantaneously. As shown in FIG. 3D, arrays of microdroplets will form mini-hydrogels that adhere to a glass or polystyrene surface upon washing with PBS.

The peptides/peptidomimetics are biocompatible. Stem cells (mesenchymal, progenitor, embryonic and induced pluripotent stem cells) and primary cells isolated from patient samples (fibroblasts, nucleus pulposus) can be mixed with the peptide during the dispensing process (FIG. 4). Following gelation, the cells are immobilized to the drop. Nanoparticles, small molecule drugs, oligonucleotides, and proteins can be similarly co-encapsulated (FIGS. 4 and 5).

Coupled with the advent of high-throughput histological screening using slide scanners, this technology can be used to evaluate different test compounds using minimal cell numbers on a single microscope slide (FIG. 6).

By incorporating cross-linkers, we can improve the mechanical stability of these mini-hydrogels. Bioactive functionalities can be also incorporated through mixing or cross-linking with polymers (FIG. 7).

We can mix different peptides/peptidomimetics/conjugates without compromising their propensity for self-assembly. This allows us to combine different compounds to access different functional groups for conjugation and vary the bulk properties.

Extending the technology towards 3D microdroplet printing and moulding, biological, organotypic constructs with distinct, multi-functional micro-niches can be obtained (FIG. 8). Multi-cellular constructs can also be obtained as the hydrogel can spatially confine different cell types during the printing process. The peptide/peptidomimetic/conjugate scaffold will provide the co-encapsulated cells with mechanical stability. Genes, small molecules and growth factors can be co-delivered to enhance cell survival, promote stem cell differentiation and modulate the host immune response. The resulting 3D biological constructs can be used as organoid models for screening drugs, studying cell behavior and disease progression, as well as tissue-engineered implants for regenerative medicine.

In addition to microdroplets, also obtain fibres (“noodles”) can be obtained by extruding the peptidic solution into a high concentration salt solution (FIG. 3E). Co-encapsulation of cells and bioactive moieties can be performed. The fibrous microenvironment can give rise to new applications such as woven scaffolds, aligned scaffolds and 3D patterned co-culture scaffolds.

### Key Features:

A novel class of peptides/peptidomimetics/conjugates which only consists of 2 to 7 amino acids which can self-assemble into nanofibrous scaffolds. The significantly shorter sequence implies a lower cost and ease of synthesis and purification compared to other self-assembling peptide/conjugate technologies.
An interesting mechanism of self-assembly into nanofibrous scaffolds in aqueous conditions and polar solvents. Such scaffolds can provide mechanical cues for cellular and tissue regeneration (biomimetic scaffold).
A versatile material which can be formulated in different ways. Some subclasses are stimuli-responsive, which facilitates the development of bio-printing technologies. Several subclasses demonstrate stimuli-responsive behavior which can be exploited for various applications.
A subclass of peptides demonstrates salt and pH-responsive gelation. In particular, instantaneous gelation can be obtained upon exposure to a physiologically compatible salt solution.
When dissolved in water, the peptidic solution has low viscosity and can be easily dispensed through needles and print-heads. This minimizes the possibility of clogging.
The stimuli-responsiveness can also be exploited to generate hydrogel fibers/′ noodles′. These fibers can subsequently be aligned or woven to create innovative scaffolds for tissue engineering and disease models.
On a macroscale, we can also use moulds (such as those made of silicone) to pattern the hydrogels in a 3D fashion.
The hydrogels are biocompatible and can be used to encapsulate cells. Upon gelation, the resulting hydrogel is stable and not easily dissociated. Therefore, encapsulated cells cannot escape.
Bioactive moieties, such as oligonucleotides, proteins and small molecule drugs, as well as nano- and microparticles, can be co-encapsulated to influence cell behavior. Drug release can also be modulated by porosity and various molecular interactions.
Post-assembly modifications are feasible due to the presence of functional groups. Large proteins such as growth factors can also be conjugated to the peptidic backbone or functional groups on the conjugate to modulate biological behavior.

### Examples

Experiments have been performed to illustrate the technical aspects of exemplary embodiments of the present invention. The following examples are described in the Experimental Methods and Results. The skilled artisan will readily recognize that the examples are intended to be illustrative and are not intended to limit the scope of the present invention.

### Experimental Methods and Results

### Circular Dichroism (CD) Spectroscopy

Secondary peptide structures were analyzed by measuring ellipticity spectra using the Aviv Circular Dichroism Spectrometer, model 410. CD samples were prepared by diluting stock peptides solutions (5-10 mg/ml) in water. The diluted peptide solutions were filled in to a cuvette with 1 mm path length and spectra were acquired. As a blank reference water was used and the reference was subtracted from the raw data before molar ellipticity was calculated. The calculation was based on the formula: [θ]_{λ}=θ_{obs}×1/(10 Lcn), where [θ]_{λ□} is the molar ellipticity at λ in deg cm² d/mol, is the observed ellipticity at □λ in mdeg, L is the path length in cm, c is the concentration of the peptide in M, and n is the number of amino acids in the peptide. Secondary structure analysis was done using CDNN software.

### Environmental Scanning Electron Microscopy (ESEM)

Samples were placed onto a sample holder of FEI Quanta 200 Environmental Scanning Electron Microscopy. The surface of interest was then examined using accelerating voltage of 10 kV at a temperature of 4° C.

### Field Emission Scanning Electron Microscopy (FESEM)

Samples were frozen at −20° C. and subsequently to −80° C. Frozen samples were further freeze dried. Freeze dried samples were fixed onto a sample holder using conductive tape and sputtered with platinum from both the top and the sides in a JEOL JFC-1600 High Resolution Sputter Coater. The coating current used was 30 mA and the process lasted for 60 sec. The surface of interest was then examined with a JEOL JSM-7400F Field Emission Scanning Electron Microscopy system using an accelerating voltage of 5-10 kV.

### Preparation of Hydrogel Droplets

We obtained hydrogel arrays by simply dispensing small volume droplets (0.5, 1, 2, 5, 10 and 20 μL) of peptide solution and subsequently mixing or washing with PBS. The viscosity and rigidity increases significantly upon gelation, conferring high shape fidelity, which enables us to localize the hydrogel droplets to the site of deposition, control the internal composition and suspend encapsulated cells or bioactive moieties, two important criteria for bioinks. To date, we have generated hydrogel droplet arrays of various volumes, encapsulating small molecules, DNA, mRNA, nanoparticles, proteins and cells.

### Encapsulation of Human Mesenchymal Stem Cells

Human mesenchymal stem cells were obtained from Lonza (Basel, Switzerland) and cultured in α-MEM medium with 20% fetal bovine serum, 2% L-glutamine and 1% penicillin-streptomycin. Upon trypsinization, the cells were suspended in PBS and subsequently added into or onto peptide solutions (in PBS). The constructs were then allowed to gel at 37° C. for 15 minutes before media was added.

Hydrophobic Peptides which Self-Assemble into Nanofibrous Hydrogels

Materials.

All peptides used in this study were manually synthesized by American Peptide Company (Sunnyvale, Calif.) using solid phase peptide synthesis and purified to >95% via HPLC. Amino acid and peptide content analysis were performed.

Preparation of Hydrogels.

To prepare the peptide hydrogels, the lyophilized peptide powders were first dissolved in milliQ water and mixed by vortexing for 30 seconds to obtain a homogenous solution. The gelation occurred between minutes to overnight, depending on the peptide concentration. Gelation can be facilitated by sonication or heating.

Functionalization of C-Terminus.

To functionalize the C-terminus, biotin and L-DOPA was incorporated during solid phase peptide synthesis by first reacting the Fmoc protected precursor to the Wang or Rink-amide resin. The final product was purified using HPLC/MS, lyophilized and evaluated for gelation.

Field Emission Scanning Electron Microscopy.

Hydrogel samples were flash frozen in liquid nitrogen and subsequently freeze-dried. Lyophilized samples were sputtered with platinum in a JEOL JFC-1600 High Resolution Sputter Coater. Three rounds of coating were performed at different angles to ensure complete coating. The coated sample was then examined with a JEOL JSM-7400F FESEM system using an accelerating voltage of 2-5 kV.

The listing or discussion of a previously published document in this specification should not necessarily be taken as an acknowledgement that the document is part of the state of the art or is common general knowledge. All documents listed are hereby incorporated herein by reference in their entirety for all purposes.

Exemplary embodiments of the invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A hydrophobic peptide and/or peptidomimetic capable of forming a hydrogel, the hydrophobic peptide and/or peptidomimetic having the general formula:
Z—(X)ₐ—Z′_{b}
wherein
Z is an N-terminal protecting group;
X is, at each occurrence, independently selected from the group consisting of aliphatic D- or L-amino acids and aliphatic D- or L-amino acid derivatives, and wherein the overall hydrophobicity decreases from N- to C-terminus;
a is an integer selected from 2 to 7, preferably 2 to 6;
Z′ is a C-terminal group; and
b is 0 or 1.

2.-3. (canceled)

4. The hydrophobic peptide and/or peptidomimetic according to claim 1, wherein all or a portion of said aliphatic amino acids are arranged in an order of decreasing amino acid size in the direction from N- to C-terminus, wherein the size of the aliphatic amino acids is defined as I=L>V>A>G.

5. The hydrophobic peptide and/or peptidomimetic according to claim 1, wherein the first N-terminal amino acid of said aliphatic amino acids is G, V or A.

6. (canceled)

7. The hydrophobic peptide and/or peptidomimetic according to claim 1, wherein said aliphatic amino acids have a sequence selected from ILVAG (SEQ ID NO: 1),
ILVAG (SEQ ID NO: 1),
LIVAG (SEQ ID NO: 2),
IVAG (SEQ ID NO: 3),
LVAG (SEQ ID NO: 4),
ILVA (SEQ ID NO: 5),
LIVA (SEQ ID NO: 6),
IVG (SEQ ID NO: 13),
VIG (SEQ ID NO: 14),
IVA (SEQ ID NO: 15),
VIA (SEQ ID NO: 16),
VI (SEQ ID NO: 17) and
IV (SEQ ID NO: 18),
wherein, optionally, there is an G, V or A preceding such sequence at the N-terminus, such as
AIVAG (SEQ ID NO. 7),
GIVAG (SEQ ID NO. 8),
VIVAG (SEQ ID NO. 9),
ALVAG (SEQ ID NO. 10),
GLVAG (SEQ ID NO. 11),
VLVAG (SEQ ID NO. 12), and/or wherein (X)ₐ has a sequence selected from the group consisting of SEQ ID NOs. 1 to 18, preferably the sequence with SEQ ID NO: 1 or SEQ ID NO. 2.

8. (canceled)

9. The hydrophobic peptide and/or peptidomimetic according to claim 1, wherein said N-terminal protecting group Z has the general formula —C(O)—R, wherein R is selected from the group consisting of H, unsubstituted or substituted alkyls, and unsubstituted or substituted aryls,
wherein R is preferably selected from the group consisting of methyl, ethyl, propyl, isopropyl, butyl and isobutyl.

10-11. (canceled)

12. The hydrophobic peptide and/or peptidomimetic according to claim 1, wherein said C-terminal group Z′ is selected from the group of small molecules, functional groups and linkers.

13-18. (canceled)

19. A composition or mixture comprising
(a) at least one hydrophobic peptide and/or peptidomimetic of claim 1, and
(b) at least one hydrophobic peptide and/or peptidomimetic capable of forming a hydrogel, the hydrophobic peptide and/or peptidomimetic having the general formula:
Z—(X)ₐ—N′_{b}
wherein
Z is as defined in claim 1;
X is as defined in claim 1;
a is as defined in claim 1;
N′ is a non-polar C-terminal group which differs from Z′, the polar C-terminal group as defined in claim 1;
and is preferably carboxylic acid, amide, alcohol, biotin, maleimide, sugars, and hydroxyacids,
and
b is 0 or 1.

20. A hydrogel comprising the hydrophobic peptide and/or peptidomimetic of claim 1.

21. The hydrogel according to claim 20, wherein the hydrogel is stable in aqueous solution at ambient temperature for a period of at least 7 days, preferably at least 2 to 4 weeks, more preferably at least 1 to 6 months, and/or wherein the hydrogel is characterized by a storage modulus G′ to loss modulus G″ ratio that is greater than 2, and/or wherein the hydrogel is characterized by a storage modulus G′ from 100 Pa to 80,000 Pa at a frequency in the range of from 0.02 Hz to 16 Hz, and/or wherein the hydrogel has a higher mechanical strength than collagen or its hydrolyzed form (gelatin).

22-24. (canceled)

25. The hydrogel according to claim 20, comprising
(a) at least one hydrophobic peptide and/or peptidomimetic of claim 1, and
(b) at least one hydrophobic peptide and/or peptidomimetic with a non-polar head group as defined in claim 19.

26-28. (canceled)

29. The hydrogel according to claim 20, wherein the hydrogel is comprised in at least one of a fuel cell, a solar cell, an electronic cell, a biosensing device, a medical device, an implant, a pharmaceutical composition and a cosmetic composition.

30-31. (canceled)

32. A method of preparing a hydrogel, the method comprising dissolving a hydrophobic peptide and/or peptidomimetic according to claim 1 in an aqueous solution.

33. The method of claim 32, wherein the dissolved hydrophobic peptide and/or peptidomimetic in aqueous solution is further exposed to temperature, wherein the temperature is in the range from 20° C. to 90° C., preferably from 20° C. to 70° C., and/or wherein the hydrophobic peptide and/or peptidomimetic is dissolved at a concentration from 0.01 μg/ml to 100 mg/ml, preferably at a concentration from 1 mg/ml to 50 mg/ml, more preferably at a concentration from about 1 mg/ml to about 20 mg/ml.

34. (canceled)

35. The method according to claim 32 comprising dissolving a hydrophobic peptide and/or peptidomimetic with a non-polar head group as defined in claim 19 in an aqueous solution.

36. A wound dressing or wound healing agent comprising a hydrogel of claim 20, or a surgical implant, or stent, the surgical implant or stent comprising a peptide and/or peptidomimetic scaffold, wherein the peptide and/or peptidomimetic scaffold is formed by a hydrogel according to claim 20.

37. (canceled)

38. A pharmaceutical and/or cosmetic composition and/or a biomedical device and/or electronic device comprising the hydrophobic peptide and/or peptidomimetic of claim 1, optionally further comprising the hydrophobic peptide and/or peptidomimetic with a non-polar head group as defined in claim 19 and/or a pharmaceutically active compound and/or a pharmaceutically acceptable carrier.

39-42. (canceled)

43. A kit of parts, the kit comprising a first container with a hydrophobic peptide and/or peptidomimetic according to claim 1 and a second container with an aqueous solution, optionally further comprising a third container with a hydrophobic peptide and/or peptidomimetic with a non-polar head group as defined in claim 19.

44. (canceled)

45. The kit of parts of claim 43, wherein the aqueous solution of the second container further comprises a pharmaceutically active compound and/or wherein the first and/or third container with a hydrophobic peptide and/or peptidomimetic further comprises a pharmaceutically active compound.

46. An in vitro or in vivo method of tissue regeneration comprising the steps:
(a) providing a hydrogel as defined in claim 20,
(b) exposing said hydrogel to cells which are to form regenerated tissue,
(c) allowing said cells to grow on said hydrogel.

47. The method of claim 46, which is performed in vivo, wherein, in step a), said hydrogel is provided at a place in a body where tissue regeneration is intended,
wherein said step a) is preferably performed by injecting said hydrogel at a place in the body where tissue regeneration is intended.

48. A method of treatment of a wound and for wound healing, said method comprising the step of
applying an effective amount of a hydrogel of claim 20 or a pharmaceutical composition of claim 38 to a wound.

49. A bioimaging device comprising a hydrogel of claim 20 for in vitro and/or in vivo use.

50. A 2D or 3D cell culture substrate comprising a hydrogel of claim 20.


SEQUENCE LISTING (18 sequences)

SEQ ID NO: 1 - protein, 5 aa, Artificial Sequence
  hydrophobic peptide
ILVAG

SEQ ID NO: 2 - protein, 5 aa, Artificial Sequence
  hydrophobic peptide
LIVAG

SEQ ID NO: 3 - protein, 4 aa, Artificial Sequence
  hydrophobic peptide
IVAG

SEQ ID NO: 4 - protein, 4 aa, Artificial Sequence
  hydrophobic peptide
LVAG

SEQ ID NO: 5 - protein, 4 aa, Artificial Sequence
  hydrophobic peptide
ILVA

SEQ ID NO: 6 - protein, 4 aa, Artificial Sequence
  hydrophobic peptide
LIVA

SEQ ID NO: 7 - protein, 5 aa, Artificial Sequence
  hydrophobic peptide
AIVAG

SEQ ID NO: 8 - protein, 5 aa, Artificial Sequence
  hydrophobic peptide
GIVAG

SEQ ID NO: 9 - protein, 5 aa, Artificial Sequence
  hydrophobic peptide
VIVAG

SEQ ID NO: 10 - protein, 5 aa, Artificial Sequence
  hydrophobic peptide
ALVAG

SEQ ID NO: 11 - protein, 5 aa, Artificial Sequence
  hydrophobic peptide
GLVAG

SEQ ID NO: 12 - protein, 5 aa, Artificial Sequence
  hydrophobic peptide
VLVAG

SEQ ID NO: 13 - protein, 3 aa, Artificial Sequence
  hydrophobic peptide
IVG

SEQ ID NO: 14 - protein, 3 aa, Artificial Sequence
  hydrophobic peptide
VIG

SEQ ID NO: 15 - protein, 3 aa, Artificial Sequence
  hydrophobic peptide
IVA

SEQ ID NO: 16 - protein, 3 aa, Artificial Sequence
  hydrophobic peptide
VIA

SEQ ID NO: 17 - protein, 2 aa, Artificial Sequence
  hydrophobic peptide
VI

SEQ ID NO: 18 - protein, 2 aa, Artificial Sequence
  hydrophobic peptide
IV